(19) United States
(12) Patent Application Publication
(10) Pub. No.: US 2024/0016137 A1
(43) Pub. Date: Jan. 18, 2024

(54) METHOD FOR DETERMINING TREATMENT PARAMETERS BASED ON THE GENETIC INFORMATION OF AT LEAST ONE ORGANISM IN THE AGRICULTURAL FIELD

(71) Applicant: BASF SE, Ludwigshafen am Rhein (DE)

(72) Inventors: Isabella SIEPE, Limburgerhof (DE); Kristina BUSCH, Limburgerhof (DE); Anja SIMON, Limburgerhof (DE); Stefan TRESCH, Limburgerhof (DE); Ingo MEINERS, Limburgerhof (DE); Andreas JOHNEN, Münster (DE); Holger HOFFMANN, Köln (DE); Bjoern KIEPE, Köln (DE)

(21) Appl. No.: 18/036,122

(22) PCT Filed: Nov. 12, 2021
(86) PCT No.: PCT/EP2021/081556

§ 371 Date: May 9, 2023

(30) Foreign Application Priority Data
Nov. 13, 2020 (EP) 20207378.9
May 19, 2021 (EP) 21174745.6
May 19, 2021 (EP) 21174778.7

(51) Int. Cl. A01M 7/00; A01M 21/04; C12Q 1/6895; G05D 1/00

(52) U.S. Cl. CPC A01M 7/0089; A01M 21/043; C12Q 1/6895; G05D 1/00

(57) ABSTRACT

A computer-implemented method for determining at least one of the treatment parameters selected from the group consisting of: a) at least one time window for a treatment in an agricultural field, b) at least one method for a treatment in an agricultural field, c) at least one product for a treatment in an agricultural field, d) at least one dose rate for a treatment in an agricultural field, and e) at least one application map for conducting a zone-specific treatment in an agricultural field, comprising the following steps: (step 1) providing genetic information of at least one organism which existed or is existing or is expected to exist in the agricultural field, (step 2) at least based on the genetic information of the at least one organism, initiating and/or performing data processing in at least one database and/or data-base system containing (i) genetic information data and (ii) data related to the at least one treatment parameter, (step 3) outputting the at least one treatment parameter based on the result of the data processing.

### FIELD OF THE INVENTION

The present invention relates to a computer-implemented method for determining treatment parameters based on the genetic information of at least one organism in the agricultural field, a data processing system comprising means for carrying out such computer-implemented method, the use of such treatment parameters for controlling an agricultural equipment, and the use of such treatment parameters for treating an agricultural field.

### BACKGROUND OF THE INVENTION

In practice, the farmer or user often faces the challenge that he/she does not know the exact genetic information (e.g. mutation) of a harmful organism, a beneficial organism or an agricultural crop species, but nevertheless has to make a decision on the time window, method, product or does rate he/she would apply for controlling the harmful organism and protecting the beneficial organism or the agricultural crop species. This may lead to the problem that the product selected by the farmer or user is inappropriate or inefficient for controlling the specific mutation of the harmful organism in his/her agricultural field, which might lead to a further spread of the harmful organism and later on to severe yield losses.

Several methods to determine the genetic information of an organism are known, inter alia the nanopore sequencing technology as for example disclosed in the patent application WO2019/149626.

In view of the above problem and challenge, it was found that there is a need to improve and simplify the decision process of the farmer or user.

### SUMMARY OF THE INVENTION

In view of the above, it is an object of the present invention to provide a computer-implemented method for determining treatment parameters based on the genetic information of at least one organism in the agricultural field, which can be easily applied by a farmer or user. It is also an object of the present invention to provide a computer-implemented method for determining treatment parameters based on the genetic information of at least one organism in the agricultural field, which supports fast and efficient decision-making for a farmer or user regarding the treatment of an agricultural field. It is also an object of the present invention to provide a computer-implemented method for determining treatment parameters based on the genetic information of at least one organism in the agricultural field, which enables the recognition and quantification of resistances against certain crop protection products. It is also an object of the present invention to provide a computer-implemented method to improve the control of harmful organisms on the agricultural field. It is also an object of the present invention to provide a computer-implemented method to improve the protection or usage of beneficial organisms on the agricultural field. It is also an object of the present invention to provide a computer-implemented method to improve the quality control of beneficial organisms used as biological crop protection on the agricultural field. It is also an object of the present invention to provide a computer-implemented method to improve the yield or biomass or nutrient content or crop quality of agricultural crop plants grown on the agricultural field. It is also an object of the present invention to provide a computer-implemented method useful for the quality control regarding past or earlier treatments. It is also an object of the present invention to provide a computer-implemented method to identify changing characteristics (i.e. weakened resistance against certain harmful organisms such as fungi) of agricultural crop plants and to adapt the crop protection or field management measures accordingly.

The objects of the present invention are solved with the subject matter of the independent claims, wherein further embodiments are incorporated in the dependent claims. It should be noted that the following described aspects and examples of the invention apply for the method as well as for the data processing system, the computer program product and the computer-readable storage medium.

According to the first aspect of the present invention, the present invention relates to a computer-implemented method for determining at least one of the treatment parameters selected from the group consisting of:
a) at least one time window for a treatment in an agricultural field,
b) at least one method for a treatment in an agricultural field,
c) at least one product for a treatment in an agricultural field,
d) at least one dose rate for a treatment in an agricultural field, and
e) at least one application map for conducting a zone-specific treatment in an agricultural field,
comprising the following steps:
(step 1) providing genetic information of at least one organism which existed or is existing or is expected to exist in the agricultural field,
(step 2) at least based on the genetic information of the at least one organism, initiating and/or performing data processing in at least one database and/or database system containing
   (i) genetic information data and
   (ii) data related to the at least one treatment parameter,
(step 3) outputting the at least one treatment parameter based on the result of the data processing.

According to a preferred embodiment of the first aspect of the present invention, a database and/or database system containing (i) genetic information data and (ii) data related to the at least one treatment parameter is provided before (step 2).

According to another preferred embodiment of the first aspect of the present invention, the present invention relates to a computer-implemented method for determining at least one of the treatment parameters selected from the group consisting of:
a) at least one time window for a treatment in an agricultural field,
b) at least one method for a treatment in an agricultural field,
c) at least one product for a treatment in an agricultural field,
d) at least one dose rate for a treatment in an agricultural field, and
e) at least one application map for conducting a zone-specific treatment in an agricultural field,
comprising the following steps:
(step 1) providing genetic information of at least one organism which existed or is existing or is expected to exist in the agricultural field,
(step 2) at least based on the genetic information of the at least one organism, initiating and/or performing data processing in at least one database and/or database system containing
(i) genetic information data, wherein genetic information data contains organ-ism response data, and
(ii) data related to the at least one treatment parameter,
wherein the data processing includes the determination of the type of response of the at least one organism based on the organism response data,
(step 3) outputting the at least one treatment parameter based on the result of the data processing.

According to a preferred embodiment of the present invention, the type of the response of the at least one organism is:
(1) target-site resistance of the at least one organism to the treatment with specific treatment parameters, preferably methods or products, or
(2) non-target-site resistance of the at least one organism to the treatment with specific treatment parameters, preferably methods or products, or
(3) response of the at least one organism to the treatment with specific treatment parameters, preferably methods or products, due to at least one of the following stress factors:
a) organism nutrition deficiencies, for example plant nutrition deficiencies,
b) heat stress, for example temperature conditions higher than 30° C.,
c) cold stress, for example temperature conditions lower than 10° C.,
d) drought stress,
e) water stress, for example existence of excessive water, e.g. after heavy rains or floods,
f) exposure to excessive sun light, for example exposure to sun light causing signs of scorch, sun burn or similar signs of irradiation,
g) acidic or alkaline pH conditions in the soil with pH values lower than pH 5 and/or pH values higher than 9,
h) salt stress, for example soil salinity,
i) pollution with chemicals, for example with heavy metals, and/or
j) destructive weather conditions, for example hail, frost, damaging wind.

According to the second aspect of the present invention, the present invention relates to a computer-implemented method for determining at least one of the above treatment parameters, comprising the following steps:
(step 1) providing genetic information of at least one organism which existed or is existing or is expected to exist in the agricultural field,
(step 1a) providing
   (A) agricultural crop data comprising (aa) information about an agricultural crop species grown, sown, planned to be grown, or planned to be sown in the agricultural field, and/or (bb) information about the growth stage of such agricultural crop, or
   (B) weather and/or geographical data relating to the location of the agricultural field, or
   (C) historic treatment data relating to treatments conducted in the agricultural field in the past,
(step 2) at least based on the genetic information of the at least one organism and based on the agricultural crop data or the weather and/or geographical data or the historic treatment data, initiating and/or performing data processing in at least one database and/or database system containing
   (i) genetic information data and
   (ii) data related to the at least one treatment parameter and
   (iii) data related to agricultural crop data, or data related to weather and/or geographical data, or data related to historic treatment data
(step 3) outputting the at least one treatment parameter based on the result of the data processing.

According to another preferred embodiment of the second aspect of the present invention, the present invention relates to a computer-implemented method for determining at least one of the above treatment parameters, comprising the following steps:
(step 1) providing genetic information of at least one organism which existed or is existing or is expected to exist in the agricultural field,
(step 1a) providing
   (A) agricultural crop data comprising (aa) information about an agricultural crop species grown, sown, planned to be grown, or planned to be sown in the agricultural field, and/or (bb) information about the growth stage of such agricultural crop, or
   (B) weather and/or geographical data relating to the location of the agricultural field, or
   (C) historic treatment data relating to treatments conducted in the agricultural field in the past,
(step 2) at least based on the genetic information of the at least one organism and based on the agricultural crop data or the weather and/or geographical data or the historic treatment data, initiating and/or performing data processing in at least one database and/or database system containing
   (i) genetic information data, wherein genetic information data contains organism response data and
   (ii) data related to the at least one treatment parameter and
   (iii) data related to agricultural crop data, or data related to weather and/or geographical data, or data related to historic treatment data wherein the data processing includes the determination of the type of response of the at least one organism based on the organism response data
(step 3) outputting the at least one treatment parameter based on the result of the data processing.

According to a preferred embodiment of the second aspect of the present invention, a database and/or database system containing (i) genetic information data and (ii) data related to the at least one treatment parameter and (iii) data related to agricultural crop data, or data related to weather and/or geographical data, or data related to historic treatment data is provided before (step 2).

In the context of the present invention, the chronological order between (step 1) and (step 1a) can be: at the same time, or (step 1) before (step 1a), or (step 1a) before (step 1).

In the context of the present invention, the weather and/or geographic data are weather data and/or geographic data.

In the context of the present invention, weather data can be any data on weather, including but not limited to temperature, soil temperature, canopy temperature, humidity, precipitation, moisture, wind conditions, sunlight levels etc.

In the context of the present invention, geographic data can be any data on geography or topography, including GPS (Global Positioning System) data, elevation data, soil data etc.

In the context of the present invention, genetic information data can be any data relating to genetic information, including an identifier for the genetic information, or the genetic information as such.

In the context of the present invention, data related to agricultural crop data can also be an identifier for the agricultural crop data, or the agricultural crop data as such.

In the context of the present invention, data related to historic treatment data can also be an identifier for the historic treatment data, or the historic treatment data as such.

In the context of the present invention, historic treatment data can be preferably provided via a user interface and/or a data interface.

In the context of the present invention, data related to weather and/or geographic data can also be an identifier for weather and/or geographic data, or weather and/or geographic data as such.

According to the third aspect of the present invention, the present invention relates to a computer-implemented method for determining at least one of the above treatment parameters, comprising the following steps:
(step 1) providing genetic information of at least one organism which existed or is existing or is expected to exist in the agricultural field,
(step 1a) providing
   (A) agricultural crop data comprising (aa) information about an agricultural crop species grown, sown, planned to be grown, or planned to be sown in the agricultural field, and/or (bb) information about the growth stage of such agricultural crop, and
   (B) weather and/or geographical data relating to the location of the agricultural field,
(step 2) at least based on the genetic information of the at least one organism and based on the agricultural crop data and the weather and/or geographical data, initiating and/or performing data processing in at least one database and/or database system containing
   (i) genetic information data and
   (ii) data related to the at least one treatment parameter and
   (iii) data related to agricultural crop data, and
   (iv) data related to weather and/or geographical data,
(step 3) outputting the at least one treatment parameter based on the result of the data processing.

According to a preferred embodiment of the third aspect of the present invention, the present invention relates to a computer-implemented method for determining at least one of the above treatment parameters, comprising the following steps:
(step 1) providing genetic information of at least one organism which existed or is existing or is expected to exist in the agricultural field,
(step 1a) providing
   (A) agricultural crop data comprising (aa) information about an agricultural crop species grown, sown, planned to be grown, or planned to be sown in the agricultural field, and/or (bb) information about the growth stage of such agricultural crop, and/or
   (B) weather and/or geographical data relating to the location of the agricultural field, and/or
   (C) historic treatment data relating to treatments conducted in the agricultural field in the past,
(step 2) at least based on the genetic information of the at least one organism and based on the agricultural crop data or the weather and/or geographical data or the historic treatment data, initiating and/or performing data processing in at least one database and/or database system containing
   (i) genetic information data, wherein genetic information data contains organism response data, and
   (ii) data related to the at least one treatment parameter and
   (iii) data related to agricultural crop data, or data related to weather and/or geographical data, or data related to historic treatment data wherein the data processing includes the determination of the type of response of the at least one organism based on the organism response data,
(step 3) outputting the at least one treatment parameter based on the result of the data processing.

According to a preferred embodiment of the third aspect of the present invention, a database and/or database system containing (i) genetic information data and (ii) data related to the at least one treatment parameter and (iii) data related to agricultural crop data and (iv) data related to weather and/or geographical data is provided before (step 2).

According to a preferred embodiment of the second and third aspect of the present invention, a database and/or database system containing (i) genetic information data and (ii) data related to the at least one treatment parameter and (iii) data related to agricultural crop data, or data related to weather and/or geographical data, or data related to historic treatment data is provided before (step 2).

According to the fourth aspect of the present invention, the present invention relates to a computer-implemented method for determining at least one of the above treatment parameters, comprising the following steps:
(step 1) providing genetic information of at least one organism which existed or is existing or is expected to exist in the agricultural field,
(step 1a) providing
   (A) agricultural crop data comprising (aa) information about an agricultural crop species grown, sown, planned to be grown, or planned to be sown in the agricultural field, and/or (bb) information about the growth stage of such agricultural crop, and
   (C) historic treatment data relating to treatments conducted in the agricultural field in the past,
(step 2) at least based on the genetic information of the at least one organism and based on the agricultural crop data and the historic treatment data, initiating and/or performing data processing in at least one database and/or database system containing
   (i) genetic information data and
   (ii) data related to the at least one treatment parameter and
   (iii) data related to agricultural crop data, and
   (iv) data related to historic treatment data,
(step 3) outputting the at least one treatment parameter based on the result of the data processing.

According to a preferred embodiment of the fourth aspect of the present invention, a database and/or database system containing (i) genetic information data and (ii) data related to the at least one treatment parameter and (iii) data related to agricultural crop data and (iv) data related to historic treatment data is provided before (step 2).

According to the fifth aspect of the present invention, the present invention relates to a computer-implemented method for determining at least one of the above treatment parameters, comprising the following steps:
(step 1) providing genetic information of at least one organism which existed or is existing or is expected to exist in the agricultural field,
(step 1a) providing
   (B) weather and/or geographical data relating to the location of the agricultural field, and
   (C) historic treatment data relating to treatments conducted in the agricultural field in the past,
(step 2) at least based on the genetic information of the at least one organism and based on the weather and/or geographical data and the historic treatment data, initiating and/or performing data processing in at least one database and/or database system containing
   (i) genetic information data and
   (ii) data related to the at least one treatment parameter and
   (iii) data related to weather and/or geographical data, and
   (iv) data related to historic treatment data,
(step 3) outputting the at least one treatment parameter based on the result of the data processing.

According to a preferred embodiment of the fifth aspect of the present invention, a database and/or database system containing (i) genetic information data and (ii) data related to the at least one treatment parameter and (iii) data related to weather and/or geographical data and (iv) data related to historic treatment data is provided before (step 2).

According to the sixth aspect of the present invention, the present invention relates to a computer-implemented method for determining at least one of the above treatment parameters, comprising the following steps:
(step 1) providing genetic information of at least one organism which existed or is existing or is expected to exist in the agricultural field,
(step 1a) providing
   (A) agricultural crop data comprising (aa) information about an agricultural crop species grown, sown, planned to be grown, or planned to be sown in the agricultural field, and/or (bb) information about the growth stage of such agricultural crop, and
   (B) weather and/or geographical data relating to the location of the agricultural field, and
   (C) historic treatment data relating to treatments conducted in the agricultural field in the past,
(step 2) at least based on the genetic information of the at least one organism and based on the agricultural crop data and the weather and/or geographical data and the historic treatment data, initiating and/or performing data processing in at least one database and/or database system containing
   (i) genetic information data and
   (ii) data related to the at least one treatment parameter and
   (iii) data related to agricultural crop data, and
   (iv) data related to weather and/or geographical data, and
   (v) data related to historic treatment data
(step 3) outputting the at least one treatment parameter based on the result of the data processing.

According to a preferred embodiment of the sixth aspect of the present invention, a database and/or database system containing (i) genetic information data and (ii) data related to the at least one treatment parameter and (iii) data related to agricultural crop data and (iv) data related to weather and/or geographical data and (v) data related to historic treatment data is provided before (step 2).

According to a preferred embodiment of the present invention, the computer-implemented method of the present invention further comprises the following step before (step 1):
(step 0) taking at least one sample of the at least one organism which existed or is existing or is expected to exist in the agricultural field, conducting a genetic analysis using the at least one sample of the at least one organism, and obtaining therefrom the genetic information of the at least one organism.

According to a preferred embodiment of the present invention, the sample of the at least one organism is a real-world physical sample of the at least one organism. The sample can be taken from any medium or material containing the organism, preferably from the soil, from the straw, from the air, from water, from parts of a plant, from pollen, from seeds, from the organism as such (e.g. insects, arachnids, nematodes, mollusks), from eggs or different growth stages of the organism (e.g. eggs or larvae of insects, arachnids, nematodes, mollusks).

According to a further preferred embodiment of the present invention, the computer-implemented method of the present invention further comprises the following step before (step 1):
(step 0) taking at least one sample of the at least one organism which existed or is existing or is expected to exist in the agricultural field, conducting a genetic analysis using the at least one sample of the at least one organism, and obtaining therefrom the genetic and/or epigenetic information of the at least one organism, wherein the genetic analysis is based on at least one of the technologies selected from the group consisting of sequencing technologies—such as Sanger sequencing, next generation sequencing, pyrosequencing, nanopore sequencing, GenapSys sequencing, sequencing by ligation (SOLiD sequencing), single-molecule real-time sequencing, Ion semiconductor (Ion Torrent sequencing) sequencing, sequencing by synthesis (Illumina), combinatorial probe anchor synthesis (cPAS—BGI/MGI)—, nanopore technology, microarray technology, graphene biosensor technology, PCR (polymerase chain reaction) technology, fast PCR technology, and other DNA/RNA amplification technologies such as isothermal amplification—such as LAMP (Loop mediated amplification), RPA (Recombinase Polymerase Amplification), Nucleic Acid Sequenced Based Amplification (NASBA) and Transcription Mediated Amplification (TMA)—, as well as epigenetic analysis such as DNA methylation, DNA-Protein interaction analysis, and Chromatin accessibility analysis, as well as expression profiling, e.g. RT qPCR, RNA sequencing.

According to a further preferred embodiment of the present invention, the computer-implemented method of the present invention further comprises the following step before (step 1):
(step 0) taking at least one sample of the at least one organism which existed or is existing or is expected to exist in the agricultural field, conducting a genetic analysis using the at least one sample of the at least one organism, and obtaining therefrom the genetic information of the at least one organism, wherein the genetic analysis is based on selective genotyping or based on sequencing technologies such as nanopore technology, pyrosequencing technology and other sequencing or next-generation sequencing (NGS) technologies.

According to a further preferred embodiment of the present invention, the computer-implemented method of the present invention further comprises the following step before (step 1):
(step 0) taking at least one sample of the at least one organism which existed or is existing or is expected to exist in the agricultural field, conducting a genetic analysis using the at least one sample of the at least one organism, and obtaining therefrom the genetic in-formation of the at least one organism, wherein the genetic analysis is based on at least one of the technologies selected from the group consisting of:
   Maxam-Gilbert sequencing, Chain-termination methods, large-scale sequencing and de novo sequencing, Shotgun sequencing, High-throughput sequencing methods, Long-read sequencing methods, Single molecule real time (SMRT) sequencing, Nanopore DNA sequencing, Short-read sequencing methods, Massively parallel signature sequencing (MPSS), Colony sequencing, pyrosequencing, Illumina (Solexa) sequencing, Combinatorial probe anchor synthesis (cPAS), SOLiD sequencing, Ion Torrent semiconductor sequencing, DNA nanoball sequencing, Heliscope single molecule sequencing, Sequencing using Microfluidic Systems, Tunnelling currents DNA sequencing, Sequencing by hybridization, Sequencing with mass spectrometry, Microfluidic Sanger sequencing, Microscopy-based techniques, RNAP sequencing, In vitro virus high-throughput sequencing,
   genetic analysis based on proteome analysis, e.g. Maldi, 2d-gel, nanopore sequencing, pro-teomics, protein sequencing, immunoblotting, in-gel digestion, high-performance liquid chromatography, and mass spectrometry, peptide analysis by complementary ionization,
   genetic analysis based on metabolite and volatile profiling (metabolome analysis), e.g. HPLC/MS, LC/MS, GC/MS, Maldi, ELISA, NMR genetic analysis based on imaging analysis, e.g. hyperspectral and multi-spec-tral imaging, Maldi imaging, Raman imaging (CARS; SERS, SRS), Nano-SIMS, IR-Imaging (especially for phenotypic adaptations).

According to a further preferred embodiment of the present invention, the computer-implemented method of the present invention further comprises the following step before (step 1):
(step 0) taking at least two samples of the at least one organism which existed or is existing or is expected to exist in the agricultural field, conducting a genetic analysis using the at least two samples of the at least one organism, and obtaining therefrom the genetic information of the at least one organism, and wherein the at least two samples have been taken from at least two different locations within the agricultural field.

According to a further preferred embodiment of the present invention, the computer-implemented method of the present invention further comprises the following step before (step 1):
(step 0) taking at least two samples of the at least one organism which existed or is existing or is expected to exist in the agricultural field, conducting a genetic analysis using the at least two samples of the at least one organism, and obtaining therefrom the genetic information of the at least one organism, and wherein the at least two samples have been taken from at least two different zones within the agricultural field.

According to a further preferred embodiment of the present invention, the computer-implemented method of the present invention further comprises the following step before (step 1):
(step 0) taking at least one sample of the at least one organism which existed or is existing or is expected to exist in the agricultural field, conducting a genetic analysis using the at least one sample of the at least one organism, and obtaining therefrom the genetic information of the at least one organism, and wherein the samples have been taken from each of the zones within the agricultural field.

According to the further aspect of the present invention, the present invention relates to a computer-implemented method for determining at least one of the above treatment parameters, comprising the following steps:
(step 0) taking at least one sample of the at least one organism which existed or is existing or is expected to exist in the agricultural field, conducting a genetic analysis using the at least one sample of the at least one organism, and obtaining therefrom the genetic information of the at least one organism,
(step 1) providing genetic information of at least one organism which existed or is existing or is expected to exist in the agricultural field,
(step 1a) providing
   (A) agricultural crop data comprising (aa) information about an agricultural crop species grown, sown, planned to be grown, or planned to be sown in the agricultural field, and/or (bb) information about the growth stage of such agricultural crop, or
   (B) weather and/or geographical data relating to the location of the agricultural field, or
   (C) historic treatment data relating to treatments conducted in the agricultural field in the past,
(step 2) at least based on the genetic information of the at least one organism and based on the agricultural crop data or the weather and/or geographical data or the historic treatment data, initiating and/or performing data processing in at least one database and/or database system containing
   (i) genetic information data and
   (ii) data related to the at least one treatment parameter and
   (iii) data related to agricultural crop data, or data related to weather and/or geographical data, or data related to historic treatment data
(step 3) outputting the at least one treatment parameter based on the result of the data processing. More preferably, the genetic information data contains organism response data, and the data processing includes the determination of the type of response of the at least one organism based on the organism response data

According to the further aspect of the present invention, the present invention relates to a computer-implemented method for determining at least one of the above treatment parameters, comprising the following steps:
(step 0) taking at least one sample of the at least one organism which existed or is existing or is expected to exist in the agricultural field, conducting a genetic analysis using the at least one sample of the at least one organism, and obtaining therefrom the genetic information of the at least one organism,
(step 1) providing genetic information of at least one organism which existed or is existing or is expected to exist in the agricultural field,
(step 1a) providing
   (A) agricultural crop data comprising (aa) information about an agricultural crop species grown, sown, planned to be grown, or planned to be sown in the agricultural field, and/or (bb) information about the growth stage of such agricultural crop, or
   (B) weather and/or geographical data relating to the location of the agricultural field, or
   (C) historic treatment data relating to treatments conducted in the agricultural field in the past,
(step 2) at least based on the genetic information of the at least one organism and based on the agricultural crop data or the weather and/or geographical data or the historic treatment data, initiating and/or performing data processing in at least one database and/or database system containing
   (i) genetic information data and
   (ii) data related to the at least one treatment parameter and
   (iii) data related to agricultural crop data, or data related to weather and/or geographical data, or data related to historic treatment data
(step 3) outputting the at least one treatment parameter based on the result of the data processing,

wherein the timeframe between sample-taking (step 0) and the provision of the genetic information (step 1) is from 1 seconds to 5 days, more preferably from 1 minute to 3 days, most preferably from 5 minutes to 1 day, particularly preferably from 10 minutes to 15 hours, particularly more preferably from 15 minutes to 10 hours, particularly from 20 minutes to 10 hours, for example from 30 minutes to 5 hours. More preferably, the genetic information data contains organism response data, and the data processing includes the determination of the type of response of the at least one organism based on the organism response data

According to the further aspect of the present invention, the present invention relates to a computer-implemented method for determining at least one of the treatment parameters selected from the group consisting of:
a) at least one time window for a treatment in an agricultural field,
b) at least one method for a treatment in an agricultural field,
c) at least one product for a treatment in an agricultural field,
d) at least one dose rate for a treatment in an agricultural field, and
e) at least one application map for conducting a zone-specific treatment in an agricultural field,
comprising the following steps:
(step 1) providing genetic information of at least one harmful organism which existed or is existing or is expected to exist in the agricultural field, and providing genetic information of at least one agricultural crop plant grown, sown, planned to be grown or sown in the agricultural field,
(step 2) at least based on the genetic information of the at least one harmful organism and on the genetic information of the at least one agricultural crop plant, initiating and/or performing data processing in at least one database and/or database system containing
   (i) genetic information data and
   (ii) data related to the at least one treatment parameter,
(step 3) outputting the at least one treatment parameter based on the result of the data processing. More preferably, the genetic information data contains organism response data, and the data processing includes the determination of the type of response of the at least one organism based on the organism response data

According to the further aspect of the present invention, the present invention relates to a computer-implemented method for determining at least one of the treatment parameters selected from the group consisting of:
a) at least one time window for a treatment in an agricultural field,
b) at least one method for a treatment in an agricultural field,
c) at least one product for a treatment in an agricultural field,
d) at least one dose rate for a treatment in an agricultural field, and
e) at least one application map for conducting a zone-specific treatment in an agricultural field,
comprising the following steps:
(step 1) providing genetic information of at least one beneficial organism which existed or is existing or is expected to exist in the agricultural field, and providing genetic information of at least one agricultural crop plant grown, sown, planned to be grown or sown in the agricultural field,
(step 2) at least based on the genetic information of the at least one beneficial organism and on the genetic information of the at least one agricultural crop plant, initiating and/or performing data processing in at least one database and/or database system containing
   (i) genetic information data and
   (ii) data related to the at least one treatment parameter,
(step 3) outputting the at least one treatment parameter based on the result of the data processing. More preferably, the genetic information data contains organism response data, and the data processing includes the determination of the type of response of the at least one organism based on the organism response data.

According to the further aspect of the present invention, the present invention relates to a computer-implemented method for determining at least one of the treatment parameters selected from the group consisting of:
a) at least one time window for a treatment in an agricultural field,
b) at least one method for a treatment in an agricultural field,
c) at least one product for a treatment in an agricultural field,
d) at least one dose rate for a treatment in an agricultural field, and
e) at least one application map for conducting a zone-specific treatment in an agricultural field,
comprising the following steps:
(step 0) taking at least one sample containing both a part of at least one harmful organism which existed or is existing or is expected to exist in the agricultural field and a part of at least one agricultural crop plant grown, sown, planned to be grown or sown in the agricultural field, conducting a genetic analysis using this at least one sample, and obtaining therefrom the genetic information of the at least one harmful organism and the genetic information of the at least one agricultural crop plant,
(step 1) providing genetic information of at least one harmful organism which existed or is existing or is expected to exist in the agricultural field, and providing genetic information of at least one agricultural crop plant grown, sown, planned to be grown or sown in the agricultural field,
(step 2) at least based on the genetic information of the at least one harmful organism and on the genetic information of the at least one agricultural crop plant, initiating and/or performing data processing in at least one database and/or database system containing
   (i) genetic information data and
   (ii) data related to the at least one treatment parameter,
(step 3) outputting the at least one treatment parameter based on the result of the data processing. More preferably, the genetic information data contains organism response data, and the data processing includes the determination of the type of response of the at least one organism based on the organism response data

In some cases, it may be not sufficient to only obtain the genetic information of the harmful organism to determine the treatment parameters for a highly efficient treatment, especially if for example the agricultural crop plant has a different genetic property than expected (e.g. is an unexpected mutant or variant). In such cases, the treatment parameters for a highly efficient treatment can only be determined after the genetic information of both the harmful organism and the agricultural crop plant have been obtained. Furthermore, to make the sample-taking (sampling) process more efficient, it is preferred to take a sample containing both a part of the harmful organism and a part of the agricultural crop plant, or to take a sample containing both part of the beneficial organism and a part of the agricultural crop plant. Thus, it is more preferred to take the following samples:
a) a leaf or another part of the agricultural crop plant partially infested with a specific fungal disease,
b) a leaf or another part of the agricultural crop plant partially infested with a specific bacterial or virus disease,
c) a leaf or another part of the agricultural crop plant which also contains genetic material of an animal pest as harmful organism, e.g. eggs, larvae, body parts, body fluids, saliva, exudates, or frass of animal pests, wherein animal pests includes insects, arachnids, nematodes, mollusks, birds or rodents and preferably includes insects, arachnids, nematodes, and mollusks, and most preferably includes insects,
d) a leaf or another part of the agricultural crop plant which also contains genetic material of a beneficial animal, e.g. eggs, larvae, body parts, body fluids, saliva, exudates, or frass of a beneficial animal, wherein beneficial animals includes insects, arachnids, nematodes, mollusks, birds or rodents and preferably includes insects, arachnids, nematodes, and mollusks and most preferably includes pollinators such as bees, butterflies, pollen wasps and flower beetles,
e) a leaf or another part of the agricultural crop plant partially infested with a specific fungal disease, which also contains genetic material of an animal pest as harmful organism, e.g. eggs, larvae, body parts, body fluids, saliva, exudates, or frass of animal pests, wherein animal pests includes insects, arachnids, nematodes, mollusks, birds or rodents and preferably includes insects, arachnids, nematodes, and mollusks, and most preferably includes insects,
f) a leaf or another part of the agricultural crop plant partially infested with a specific fungal disease, which also contains genetic material of a beneficial animal, e.g. eggs, larvae, body parts, body fluids, saliva, exudates, or frass of a beneficial animal, wherein beneficial animals includes insects, arachnids, nematodes, mollusks, birds or rodents and preferably includes insects, arachnids, nematodes, and mollusks and most preferably includes pollinators such as bees, butterflies, pollen wasps and flower beetles.

In a further preferred embodiment, the beneficial organism includes predatory insects used for pest management.

Regarding animal pests (including insects, arachnids, nematodes, mollusks, birds or rodents), the sampling method is preferably selected according to the species of the animal pests.

As an example, it is known that there is one common biotype of white flies and one multiple-insecticide-resistant biotype of white flies. Through a genetic analysis such as an DNA analysis (following the sampling of white flies), these two biotypes can be distinguished, thus different treatment parameters can be outputted depending on the biotype detected.

In a further preferred embodiment of (step 0), if the organism is an animal (such as insects, arachnids, nematodes, mollusks, birds or rodents), the animal is not sampled as such, but sampled in the form of e.g. eggs, larvae, body parts, body fluids, saliva, exudates, frass, metabolic substances, metabolic profiles, hormones and pheromones of the organism. Regarding the different life stages (eggs, larvae, adults) of an animal pest such as insects, it is preferred that the sample (used for genetic analysis) is taken from the life stage which is most useful or relevant for the pest management and/or pest control of the corresponding species of the animal pest.

In a further preferred embodiment, if a sample of an animal such as an insect, arachnid, nematode or mollusk or a sample containing its eggs, larvae, body parts, body fluids, saliva, exudates, frass, metabolic substances, metabolic profiles, hormones and pheromones has been taken, not only genetic information about such animal is obtained through genetic analysis, but also genetic information on possible harmful bacteria, viruses or viroids carried by such animal is obtained through genetic analysis based on the same sample. Such harmful bacteria, viruses or viroids may be capable of causing severe damages to the agricultural crop plants.

According to the further aspect of the present invention, the present invention relates to a computer-implemented method for determining at least one of the treatment parameters selected from the group consisting of:
a) at least one time window for a treatment in an agricultural field,
b) at least one method for a treatment in an agricultural field,
c) at least one product for a treatment in an agricultural field,
d) at least one dose rate for a treatment in an agricultural field, and
e) at least one application map for conducting a zone-specific treatment in an agricultural field,
comprising the following steps:
(step 0) taking at least one sample containing both a part of at least one beneficial organism which existed or is existing or is expected to exist in the agricultural field and a part of at least one agricultural crop plant grown, sown, planned to be grown or sown in the agricultural field, conducting a genetic analysis using this at least one sample, and obtaining therefrom the genetic information of the at least one beneficial organism and the genetic information of the at least one agricultural crop plant,
(step 1) providing genetic information of at least one beneficial organism which existed or is existing or is expected to exist in the agricultural field, and providing genetic information of at least one agricultural crop plant grown, sown, planned to be grown or sown in the agricultural field,
(step 2) at least based on the genetic information of the at least one beneficial organism and on the genetic information of the at least one agricultural crop plant, initiating and/or performing data processing in at least one database and/or database system containing
   (i) genetic information data and
   (ii) data related to the at least one treatment parameter,
(step 3) outputting the at least one treatment parameter based on the result of the data processing. More preferably, the genetic information data contains organism response data, and the data processing includes the determination of the type of response of the at least one organism based on the organism response data

According to a further preferred embodiment of the present invention, in (step 1) of the computer-implemented method, genetic information of at least one harmful organism which existed or is existing or is expected to exist in the agricultural field, and genetic information of at least one agricultural crop plant grown, sown, planned to be grown or sown in the agricultural field are provided.

According to a further preferred embodiment of the present invention, in (step 1) of the computer-implemented method, genetic information of at least one beneficial organism which existed or is existing or is expected to exist in the agricultural field, and genetic information of at least one agricultural crop plant grown, sown, planned to be grown or sown in the agricultural field are provided.

According to a further preferred embodiment of the present invention, in (step 0) of the computer-implemented method, at least one sample containing both a part of at least one harmful organism which existed or is existing or is expected to exist in the agricultural field and a part of at least one agricultural crop plant grown, sown, planned to be grown or sown in the agricultural field is taken, a genetic analysis using this at least one sample is conducted, and therefrom the genetic information of the at least one harmful organism and the genetic information of the at least one agricultural crop plant are obtained.

According to a further preferred embodiment of the present invention, in (step 0) of the computer-implemented method, at least one sample containing both a part of at least one beneficial organism which existed or is existing or is expected to exist in the agricultural field and a part of at least one agricultural crop plant grown, sown, planned to be grown or sown in the agricultural field is taken, a genetic analysis using this at least one sample is conducted, and therefrom the genetic information of the at least one beneficial organism and the genetic information of the at least one agricultural crop plant are obtained.

According to a further preferred embodiment of the present invention, in (step 0) of the computer-implemented method, at least one sample containing both a part of at least one harmful organism which existed or is existing or is expected to exist in the agricultural field and a part of at least one agricultural crop plant grown, sown, planned to be grown or sown in the agricultural field is taken, a genetic analysis using this at least one sample is conducted, and therefrom the genetic information of the at least one harmful organism and the genetic information of the at least one agricultural crop plant are obtained, and in (step 1) of the computer-implemented method, genetic information of at least one harmful organism which existed or is existing or is expected to exist in the agricultural field, and genetic information of at least one agricultural crop plant grown, sown, planned to be grown or sown in the agricultural field are provided.

According to a further preferred embodiment of the present invention, in (step 0) of the computer-implemented method, at least one sample containing both a part of at least one beneficial organism which existed or is existing or is expected to exist in the agricultural field and a part of at least one agricultural crop plant grown, sown, planned to be grown or sown in the agricultural field is taken, a genetic analysis using this at least one sample is conducted, and therefrom the genetic information of the at least one beneficial organism and the genetic information of the at least one agricultural crop plant are obtained, and in (step 1) of the computer-implemented method, genetic information of at least one beneficial organism which existed or is existing or is expected to exist in the agricultural field, and genetic information of at least one agricultural crop plant grown, sown, planned to be grown or sown in the agricultural field are provided.

According to a further preferred embodiment of the present invention, the genetic analysis of the at least one organism is conducted using a portable device operated in the agricultural field.

According to a further preferred embodiment of the present invention, the genetic analysis of the at least one organism is conducted in a facility outside the agricultural field.

According to a further preferred embodiment of the present invention, the timeframe between sample-taking and the provision of the genetic information is from 1 seconds to 5 days, more preferably from 1 minute to 3 days, most preferably from 5 minutes to 1 day, particularly preferably from 10 minutes to 15 hours, particularly more preferably from 15 minutes to 10 hours, particularly from 20 minutes to 10 hours, for example from 30 minutes to 5 hours.

According to a further preferred embodiment of the present invention, the genetic information of the at least one organism has been provided by a user interface and/or by a data interface.

According to a further preferred embodiment of the present invention, the at least one organism is a harmful organism selected from the group consisting of: weeds, fungi, viruses, viroids, bacteria, insects, arachnids, nematodes, mollusks, birds, and rodents.

According to a further preferred embodiment of the present invention, the at least one organism is a beneficial organism selected from the group consisting of: beneficial plants, fungi, viruses, viroids, bacteria, insects, arachnids, nematodes, mollusks, birds, rodents, and protozoa.

According to a further preferred embodiment of the present invention, the at least one organism is an agricultural crop species grown, sown, planned to be grown, or planned to be sown in the agricultural field.

According to a further preferred embodiment of the present invention, a first treatment using at least one of the treatment parameters with or without the use of genetic information has been already carried out in the timeframe of not more than 30 days ago, more preferably in the timeframe of not more than 20 days ago, most preferably in the timeframe of not more than 10 days ago, for example in the timeframe of not more than 5 days ago.

According to a further preferred embodiment of the present invention, at least the steps (step 1), (step 2) and (step 3) are carried out in a real-time mode, i.e. preferably less than a minute, more preferably within 10 to 45 seconds, most preferably within 1 to 10 seconds, more preferably within 0.5 to 1 seconds, most preferably within 100 to 500 milliseconds, particularly within 10 to 100 milliseconds.

According to a further preferred embodiment of the present invention, at least the steps (step 1), (step 2) and (step 3) and the further step of outputting the determined treatment parameter as a control signal (or control file) for an agricultural equipment are carried out in a real-time mode, i.e. preferably less than a minute, more preferably within 10 to 45 seconds, most preferably within 1 to 10 seconds, more preferably within 0.5 to 1 seconds, most preferably within 100 to 500 milliseconds, particularly within 10 to 100 milliseconds.

According to a further preferred embodiment of the present invention, at least the steps (step 0), and (step 1), and (step 2), and (step 3) are carried out in a real-time mode, i.e. preferably less than ten minutes, more preferably less than five minutes, most preferably less than two minutes, particularly more preferably within 10 to 45 seconds, particularly most preferably within 1 to 10 seconds, particularly within 0.5 to 1 seconds, for instance within 100 to 500 milliseconds, for example within 10 to 100 milliseconds. For this preferred embodiment, genetic analysis based on imaging can be preferably used in (step 0).

According to a further preferred embodiment of the present invention, at least the steps (step 0), and (step 1), and (step 2), and (step 3), and the further step of outputting the determined treatment parameter are carried out in a real-time mode, i.e. preferably less than ten minutes, more preferably less than five minutes, most preferably less than two minutes, particularly more preferably within 10 to 45 seconds, particularly most preferably within 1 to 10 seconds, particularly within 0.5 to 1 seconds, for instance within 100 to 500 milliseconds, for example within 10 to 100 milliseconds. For this preferred embodiment, genetic analysis based on imaging can be preferably used in (step 0).

According to a further preferred embodiment of the present invention, the treatment parameter will be outputted as a control signal for an agricultural equipment.

In another preferred embodiment (referred to as “real-time-treatment-control-based-on-genetic-analysis embodiment”), the following computer-implemented method was found: Computer-implemented method for determining at least one of the treatment parameters selected from the group consisting of:
a) at least one time window for a treatment in an agricultural field,
b) at least one method for a treatment in an agricultural field,
c) at least one product for a treatment in an agricultural field,
d) at least one dose rate for a treatment in an agricultural field, and
e) at least one application map for conducting a zone-specific treatment in an agricultural field,
comprising the following steps:
(step 0) taking at least one sample of the at least one organism which existed or is existing or is expected to exist in the agricultural field, conducting a genetic anal-ysis using the at least one sample of the at least one organism, and obtaining therefrom the genetic information of the at least one organism, wherein the genetic analysis of the at least one organism is conducted using a portable device operated in the agricultural field,
(step 1) providing genetic information of at least one organism which existed or is existing or is expected to exist in the agricultural field,
(step 2) at least based on the genetic information of the at least one organism, initiating and/or performing data processing in at least one database and/or database system containing
(i) genetic information data, wherein genetic information data contains organ-ism response data, and
(ii) data related to the at least one treatment parameter,
wherein the data processing includes the determination of the type of response of the at least one organism based on the organism response data, (step 3) outputting the at least one treatment parameter based on the result of the data processing, wherein this at least one treatment parameter is outputted as a control signal for an agricultural equipment,
wherein the steps (step 0) and (step 1) and (step 2) and (step 3) are carried out in a realtime mode, i.e. preferably less than ten minutes, more preferably less than five minutes, most preferably less than two minutes, particularly more preferably within 10 to 45 seconds, particularly most preferably within 1 to 10 seconds, particularly within 0.5 to 1 seconds, for instance within 100 to 500 milliseconds, for example within 10 to 100 milliseconds.

In another preferred embodiment (referred to as “real-time-treatment-conducting-based-on-genetic-analysis embodiment”), the following computer-implemented method was found: Computer-implemented method for determining at least one of the treatment parameters selected from the group consisting of:
a) at least one time window for a treatment in an agricultural field,
b) at least one method for a treatment in an agricultural field,
c) at least one product for a treatment in an agricultural field,
d) at least one dose rate for a treatment in an agricultural field, and
e) at least one application map for conducting a zone-specific treatment in an agricultural field,
comprising the following steps:
(step 0) taking at least one sample of the at least one organism which existed or is existing or is expected to exist in the agricultural field, conducting a genetic anal-ysis using the at least one sample of the at least one organism, and obtaining therefrom the genetic information of the at least one organism, wherein the genetic analysis of the at least one organism is conducted using a portable device operated in the agricultural field,
(step 1) providing genetic information of at least one organism which existed or is existing or is expected to exist in the agricultural field,
(step 2) at least based on the genetic information of the at least one organism, initiating and/or performing data processing in at least one database and/or database system containing
(i) genetic information data, wherein genetic information data contains organ-ism response data, and
(ii) data related to the at least one treatment parameter,
wherein the data processing includes the determination of the type of response of the at least one organism based on the organism response data,
(step 3) outputting the at least one treatment parameter based on the result of the data processing, wherein this at least one treatment parameter is outputted as a control signal for an agricultural equipment,
(step 4) conducting the treatment on the agricultural field through the agricultural equipment according to the outputted at least one treatment parameter,

wherein the steps (step 0) and (step 1) and (step 2) and (step 3) and (step 4) are carried out in a real-time mode, i.e. preferably less than ten minutes, more preferably less than five minutes, most preferably less than two minutes, particularly more preferably within 10 to 45 seconds, particularly most preferably within 1 to 10 seconds, particularly within 0.5 to 1 seconds, for instance within 100 to 500 milliseconds, for example within 10 to 100 milliseconds.

According to a further preferred embodiment of the present invention, the present invention also relates to a data processing system comprising means for carrying out the computer-implemented method of this invention.

According to a further preferred embodiment of the present invention, the present invention also relates to a computer program product comprising instructions which, when the program is executed by a computer, cause the computer to carry out the computer-implemented method of the invention

According to a further preferred embodiment of the present invention, the present invention also relates to a computer-readable storage medium comprising instructions which, when executed by a computer, cause the computer to carry out the computer-implemented method according to the invention.

According to a further preferred embodiment of the present invention, the present invention also relates to the use of the treatment parameters determined by the computer-implemented method according to the invention for controlling an agricultural equipment.

According to a further preferred embodiment of the present invention, the present invention also relates to the use of the treatment parameters determined by the computer-implemented method according to the invention for treating an agricultural field.

In the context of the present invention, the term “organism” is understood to be any kind of individual entities having the properties of life, including but not limited to plants, crop plants, weeds, fungi, viruses, viroids, bacteria, insects, arachnids, nematodes, mollusks, birds, rodents, other animals, protozoa, protists, and archaea.

In the context of the present invention, the term “harmful organism” is understood to be any organism which has a negative impact to the growth or to the health of the agricultural crop plant.

In the context of the present invention, the term “beneficial organism” is understood to be any organism which does not have a negative impact to the growth or to the health of the agricultural crop plant. The terms “beneficial organism” and “benign organism” are used synonymously.

In the context of the present invention, the term “genetic information” is understood to be any kind of information on the genetic properties of an organism, including but not limited to DNA sequence, RNA sequence, parts of DNA and/or RNA sequences, molecular structure of DNA and/or RNA, epigenetic information (e.g. methylation of DNA parts), information on gene mutations, information on gene copy number variation, information on overexpression of a gene, information on expression level of a gene, information on gene shifting, information on the ratio between wild type and mutants, information on the ratio between different mutants, information on the ratio between mutants and other variants (e.g. epigenetic variants), information on the ratio of different variants (e.g. epigenetic variants), information on a type of plant disease (e.g. Septoria, yellow rust, Asian soybean rust) or other diseases. In the context of the present invention, the term “genetic information” also includes the information that certain wild types, mutants, or variants (e.g. epigenetic variants) or DNA/RNA sequences, or parts of the DNA/RNA sequences, or specific epigenetic information are absent. In the context of the present invention, the term “genetic information” also includes the information that specific genetic information is absent (e.g. that the information that a specific type of Septoria is absent is also a genetic information). In a preferred embodiment of the present invention, genetic information” is at least one of the following information: DNA sequence, RNA sequence, parts of DNA and/or RNA sequences, molecular structure of DNA and/or RNA, epigenetic information (e.g. methylation of DNA parts), information on gene mutations, information on gene copy number variation, information on overexpression of a gene, information on expression level of a gene, information on gene shifting, information on the ratio between wild type and mutants, information on the ratio between different mutants, information on the ratio between mutants and other variants (e.g. epigenetic variants), information on the ratio of different variants (e.g. epigenetic variants), information on a type of plant disease (e.g. Septoria, yellow rust, Asian soybean rust) or other diseases. In another preferred embodiment of the present invention, genetic information” is at least one of the following information: DNA sequence, RNA sequence, molecular structure of DNA and/or RNA, parts of DNA and/or RNA sequences, epigenetic information (e.g. methylation of DNA parts). In another preferred embodiment of the present invention, genetic information” is at least one of the following information: DNA sequence, RNA sequence. In another preferred embodiment of the present invention, genetic information” is at least one of the following information: information on gene mutations, information on gene copy number variation, information on overexpression of a gene, information on expression level of a gene, information on gene shifting, information on the ratio between wild type and mutants, information on the ratio between different mutants, information on the ratio between mutants and other variants (e.g. epigenetic variants), information on the ratio of different variants (e.g. epigenetic variants), information on a type of plant disease (e.g. Septoria, yellow rust, Asian soybean rust) or other diseases. In another preferred embodiment of the present invention, genetic information” is at least one of the following information: information on gene mutations, information on gene copy number variation, information on overexpression of a gene, information on expression level of a gene, information on gene shifting. In another preferred embodiment of the present invention, genetic information” is at least one of the following information: information on the ratio between wild type and mutants, information on the ratio between different mutants, information on the ratio between mutants and other variants (e.g. epigenetic variants), information on the ratio of different variants (e.g. epigenetic variants).

In another preferred embodiment of the present invention, the term “genetic information” also includes the information on the metabolism of an organism and on the abiotic or biotic stress response (including phenotypic adaptation such as thicker cuticula) of an organism as far as such information is related or correlated to a mutation, biotype, genetic variant, or epigenetic variant of the organism.

In another preferred embodiment of the present invention, the genetic information is the information on the resistance of an organism against certain crop protection products.

In the context of the present invention, the term “data processing” is understood to be any operation on the data to produce or output meaningful information, which is conducted by a computer system. Data processing includes but is not limited to data validation, data analysis, data aggregation, data sorting, data classification, data summarization, data conversion, data modification, data update etc. Data processing in a database or database system also may include the automated request in a database or database system and the automated outputting of the result of such request.

In the context of the present invention, the term “database” is understood to be any organized collection of data, which can be stored and accessed electronically from a computer system, including but not limited to relational database, non-relational database, graph database, network database, cloud database, in-memory database, active database, data warehouse, deductive database, distributed database, embedded database, end-user database, hypertext or hypermedia database, knowledge database, mobile database, operational database, parallel database, probabilistic database, real-time database, spatial database, temporal database, terminology-oriented database, and Excel databases. In a preferred embodiment of the present invention, the database is at least one of the following databases: relational database, nonrelational database, graph database, network database, cloud database, in-memory database, active database, data warehouse, deductive database, distributed database, embedded database, end-user database, hypertext or hypermedia database, knowledge database, mobile database, operational database, parallel database, probabilistic database, real-time database, spatial database, temporal database, terminology-oriented database, and Excel databases.

Preferably, the database comprises information on resistance of organisms, especially their genetic or epigenetic variants, mutants etc., against specific treatment parameters such as crop protection products. Such information on resistances can be extracted from resistance databases such as FRAC, IRAC, HRAC, or weedscience.org databases.

In the context of the present invention, the term “database system” is understood to be a system comprising more than one database which are connected to each other, including but not limited to federated database systems, array database management systems, and other database management systems.

In the context of the present invention, the term “treatment” is understood to be any kind of treatment possible on an agricultural field, including but not limited to seeding, fertilization, crop protection, growth regulation, harvesting, adding or removing of organisms—particularly crop plants—, as well as soil treatment, soil nutrient management, soil nitrogen management, tilling, ploughing, irrigation. In a preferred embodiment of the present invention, treatment is one of the following activities: seeding, fertilization, crop protection, growth regulation, harvesting, adding or removing of organisms—particularly crop plants—, as well as soil treatment, soil nutrient management, soil nitrogen management, tilling, ploughing, irrigation. In another preferred embodiment of the present invention, treatment is seeding. In another preferred embodiment of the present invention, treatment is fertilization. In another preferred embodiment of the present invention, treatment is crop protection. In another preferred embodiment of the present invention, treatment is growth regulation. In another preferred embodiment of the present invention, treatment is harvesting. In another preferred embodiment of the present invention, treatment is adding or removing of organisms—particularly crop plants.

In the context of the present invention, the term “agricultural field” is understood to be any area in which organisms, particularly crop plants, are produced, grown, sown, and/or planned to be produced, grown or sown. The term “agricultural field” also includes horticultural fields, silvicultural fields and fields for the production and/or growth of aquatic organisms.

In the context of the present invention, the term “treatment parameter” is any parameter useful for a treatment in an agricultural field and is selected from the group consisting of:
a) at least one time window for a treatment in an agricultural field,
b) at least one method for a treatment in an agricultural field,
c) at least one product for a treatment in an agricultural field,
d) at least one dose rate for a treatment in an agricultural field, and
e) at least one application map for conducting a zone-specific treatment in an agricultural field.

According to a further preferred embodiment of the present invention, the treatment parameter is a time window for a treatment in an agricultural field.

According to a further preferred embodiment of the present invention, the treatment parameter is a method for a treatment in an agricultural field.

According to a further preferred embodiment of the present invention, the treatment parameter is a product for a treatment in an agricultural field.

According to a further preferred embodiment of the present invention, the treatment parameter is a dose rate for a treatment in an agricultural field.

According to a further preferred embodiment of the present invention, the treatment parameter is an application map for conducting a zone-specific treatment in an agricultural field.

In the context of the present invention, a “method for a treatment” includes but is not limited to
mechanical methods—e.g. mechanical weed removal by machinery such as robots, which for example cuts out the weed,
physical methods—e.g. weed removal by optical light such as laser,
chemical methods—e.g. weed removal by spraying a herbicide, or e.g. attracting beneficial insects to another area outside the agricultural field using chemical attractants,
biological methods—e.g. weed removal by applying a microorganism used as bioherbicide, or e.g. attracting beneficial insects to another area outside the agricultural field by placing other organisms (which serves as food for the beneficial insects) into this another area.

In the context of the present invention, the term “product” is understood to be any object or material useful for the treatment. In the context of the present invention, the term “product” includes but is not limited to:
chemical products such as fungicide, herbicide, insecticide, acaricide, molluscicide, nematicide, avicide, piscicide, rodenticide, repellant, bactericide, biocide, safener, plant growth regulator, urease inhibitor, nitrification inhibitor, denitrification inhibitor, or any combination thereof.
biological products such as microorganisms useful as fungicide (biofungicide), herbicide (bioherbicide), insecticide (bioinsecticide), acaricide (bioacaricide), molluscicide (biomolluscicide), nematicide (bionematicide), avicide, piscicide, rodenticide, repellant, bactericide, biocide, safener, plant growth regulator, urease inhibitor, nitrification inhibitor, denitrification inhibitor, or any combination thereof.
fertilizer and nutrient,
seed and seedling,
water,
further non-chemical products such as mechanical/physical/optical weed or fungi or insect removal equipment, including weed or fungi or insect removal machines, robots or drones, and
any combination thereof.

In the context of the present invention, the term “product” also includes a combination of different products.

In a preferred embodiment of the present invention, product is at least one chemical product selected from: fungicide, herbicide, insecticide, acaricide, molluscicide, nematicide, avicide, piscicide, rodenticide, repellant, bactericide, biocide, safener, plant growth regulator, urease inhibitor, nitrification inhibitor, denitrification inhibitor; or any combination thereof.

In another preferred embodiment of the present invention, product is at least one biological product selected from: microorganisms useful as fungicide, herbicide, insecticide, acaricide, molluscicide, nematicide, avicide, piscicide, rodenticide, repellant, bactericide, biocide, safener, plant growth regulator, urease inhibitor, nitrification inhibitor, denitrification inhibitor; or any combination thereof.

In another preferred embodiment of the present invention, product is fertilizer and/or nutrient.

In another preferred embodiment of the present invention, product is seed and/or seedling.

In the context of the present invention, the term “dose rate” is understood as amount of product to be applied per area, for example expressed as liter per hectare (L/ha).

In the context of the present invention, the time window for a treatment can preferably range 5 from 10 days to 1 hour, more preferably from 7 days to 3 hours, most preferably from 5 days to hours, particularly preferably from 3 days to 8 hours, particularly more preferably from 2 days to 12 hours, particularly from 36 hours to 16 hours, for example from 28 hours to 20 hours.

In the context of the present invention, the term “application map” is understood to be a map indicating a two-dimensional spatial distribution of the amounts, or dose rates, or types, or forms of products which should be applied on different locations or zones within an agricultural field. In the context of the present invention, the term “zone” is understood to be a sub-field zone or a part of an agricultural field, i.e. an agricultural field can be spatially divided into more than one zone, wherein each zone may have different properties such as different biomass levels or different weed and/or pathogen infestation risks. Particularly, the application map may indicate that in different zones, different amounts, or dose rates, or types, or forms of products should be applied. For example, the application map may indicate that in the first zone, the product should be applied in a product dose rate of 10 liters per hectare, and in the second zone, the same product should be applied in a product dose rate of 20 liters per hectare.

In another preferred embodiment of the present invention, a computer-implemented method for determining at least one of the treatment parameters selected from the group consisting of
a) a ranked or unranked list of more than one time window for a treatment in an agricultural field, and/or
b) a ranked or unranked list of more than one method for a treatment in an agricultural field, and/or
c) a ranked or unranked list of more than one product for a treatment in an agricultural field, and/or
d) a ranked or unranked list of more than one dose rate for a treatment in an agricultural field, and/or
e) a ranked or unranked list of more than one application map for conducting a zone-specific treatment in an agricultural field, comprising the following steps:
(step 1) providing genetic information of at least one organism which existed or is existing or is expected to exist in the agricultural field,
(step 2) at least based on the genetic information of the at least one organism, initiating and/or performing data processing in at least one database and/or database system containing
   (i) genetic information data and
   (ii) data related to the at least one treatment parameter,
(step 3) outputting the at least one treatment parameter based on the result of the data processing,
   is provided. More preferably, the genetic information data contains organism response data, and the data processing includes the determination of the type of response of the at least one organism based on the organism response data.

The ranked or unranked list may have two, more than two, more than three, or more than four entries.

In another preferred embodiment of the present invention, a computer-implemented method for determining at least one of the treatment parameters selected from the group consisting of
a) a ranked list of more than one time window for a treatment in an agricultural field, and/or
b) a ranked list of more than one method for a treatment in an agricultural field, and/or
c) a ranked list of more than one product for a treatment in an agricultural field, and/or
d) a ranked list of more than one dose rate for a treatment in an agricultural field, and/or
e) a ranked list of more than one application map for conducting a zone-specific treatment in an agricultural field,
comprising the following steps:
(step 1) providing genetic information of at least one organism which existed or is existing or is expected to exist in the agricultural field,
(step 2) at least based on the genetic information of the at least one organism, initiating and/or performing data processing in at least one database and/or database system containing
   (i) genetic information data and
   (ii) data related to the at least one treatment parameter,
(step 3) outputting the at least one treatment parameter based on the result of the data processing,
   is provided,
wherein the ranked list is obtained via a ranking according to the expected efficacy or suitability of the treatment, preferably
   in terms of the control of a harmful organism (in case at least one organism is a harmful organism), or
   in terms of the protection or usage of beneficial organism (in case at least one organism is a beneficial organism), or
   in terms of the yield, biomass, nutrient content, crop quality or plant health of the agricultural crop plant grown or sown in the agricultural field. More preferably, the genetic information data contains organism response data, and the data processing includes the determination of the type of response of the at least one organism based on the organism response data.

In another preferred embodiment of the present invention, in case of the at least one organism being a harmful organism selected from the group consisting of weeds, fungi, viruses, viroids, bacteria, insects, arachnids, nematodes, mollusks, birds, and rodents, the data processing in (step 2) is carried out in a way to determine or output the at least one treatment parameter with the objective of achieving the most appropriate or efficient (e.g. in terms of efficacy) control of harmful organism.

In another preferred embodiment of the present invention, in case of the at least one organism being a harmful organism selected from the group consisting of weeds, fungi, viruses, viroids, bacteria, insects, arachnids, nematodes, mollusks, birds, and rodents, the data processing in (step 2) is carried out in a way to determine or output the at least one treatment parameter with the objective of achieving the most appropriate or efficient (e.g. in terms of efficacy) control of harmful organism over the long-term of e.g. 2 years to 6 years, thus considering the possible or expected development of resistances of the organisms against specific crop protection products. Information on such resistances can be extracted from resistance databases such as FRAC, IRAC, HRAC, or weedscience.org databases.

In another preferred embodiment of the present invention, in case of the at least one organism being a beneficial organism selected from the group consisting of beneficial plants, fungi, viruses, viroids, bacteria, insects, arachnids, nematodes, mollusks, birds, rodents, and protozoa, the data processing in (step 2) is carried out in a way to determine or output at least one treatment parameter with the objective of achieving the best possible and most efficient (e.g. in terms of efficacy) protection of the beneficial organism or achieving the best possible usage or growth of the beneficial organism or achieving the highest level of biodiversity.

In another preferred embodiment of the present invention, in case of the at least one organism being an agricultural crop plant, the data processing in (step 2) is carried out in a way to determine or output at least one treatment parameter with the objective of achieving the best possible and most efficient usage or growth of the agricultural crop plant, e.g. achieving the highest yield or biomass or nutrient content or crop quality.

In a preferred embodiment of the present invention, “an organism expected to exist in an agricultural field” is an organism which is expected to exist in an agricultural field according to corresponding predictions or forecasts related to such organism in this agricultural field or in its surroundings or its region or its country—such as predictions on the presence of plant diseases, insect pests or weeds—or according to corresponding historic experience related to such organism in this agricultural field or in its surroundings or its region or its country, or according to corresponding historic experience related to the growth of a specific agricultural crop plant. The predictions or forecasts related to such organism can be based on corresponding computer models. Regarding sample-taking (step 0), for example a crop leave can be sampled, and through the genetic analysis using this crop leave sample, it can be checked whether the organism expected to exist (especially a harmful organism such as fungi expected to exist) can be actually found in the crop leave sample.

Herbicide Classes

In a preferred embodiment, the product is an herbicide from at least one of the following classes: acetamides, amides, aryloxyphenoxypropionates, benzamides, benzofuran, benzoic acids, benzothiadiazinones, bipyridylium, carbamates, chloroacetamides, chlorocarboxylic acids, cyclohexanediones, dinitroanilines, dinitrophenol, diphenyl ether, glycines, imidazolinones, isoxazoles, isoxazolidinones, nitriles, N-phenylphthalimides, oxadiazoles, oxazolidinediones, oxyacetamides, phenoxycarboxylic acids, phenylcarbamates, phenylpyrazoles, phenylpyrazolines, phenylpyridazines, phosphinic acids, phosphoroamidates, phosphorodithioates, phthalamates, pyrazoles, pyridazinones, pyridines, pyridinecarboxylic acids, pyridinecarboxamides, pyrimidinediones, pyrimidinyl(thio)benzoates, quinolinecarboxylic acids, semicarbazones, sulfonylaminocarbonyltriazolinones, sulfonylureas, tetrazolinones, thiadiazoles, thiocarbamates, triazines, triazinones, triazoles, triazolinones, triazolocarboxamides, triazolopyrimidines, triketones, uracils, ureas.

Weed Species

In a preferred embodiment, the harmful organism is a monocotyledonous weed or a dicotyledonous weed.

In one embodiment, the harmful organism is selected from monocotyledonous weed species.

Preferably, the harmful organism is selected from the family Poaceae. More preferably, the harmful organism is selected from the tribes Aveneae, Bromeae, Paniceae and Poeae. In one embodiment, the harmful organism is selected from the tribe Aveneae. In another embodiment, the harmful organism is selected from the tribe Bromeae. In yet another embodiment, the harmful organism is selected from the tribe Paniceae. In still another embodiment, the harmful organism is selected from the tribe Poeae.

In particular, the compositions and methods of the present invention may be used for controlling annual weeds such as gramineous weeds (grass weeds) including, but not limited to, the genera Aegilops such as Aegilops cylindrical (AEGCY, jointed goatgrass); Agropyron such as Agropyron repens (AGRRE, common couchgrass); Alopecurus such as Alopecurus myosuroides blackgrass (ALOMY, blackgrass) or Alopecurus aequalis (ALOAE, foxtail); Apera such as Apera spica-venti (APESV, silky wind grass); Avena such as Avena fatua (AVEFA, wild oat) or Avena sterilis subsp. Sterilis (AVEST, sterile oat); Brachiaria such as Brachiaria plantaginea (BRAPL, Alexander grass) or Brachiaria decumbens (BRADC, Surinam grass); Bromus such as Bromus inermis (BROIN, awnless brome), Bromus sterilis (BROST, barren bromegrass), Bromus tectorum (BROTE, cheatgrass), Bromus arvensis (BROAV, field bromegrass), Bromus secalinus (BROSE, rye bromegrass) or Bromus hordeacus (BROMO, lopgrass); Cenchrus such as Cenchrus echinatus (CCHEC, Mossman River grass); Cynodon such as Cynodon dactylon (CYNDA, bermudagrass); Digitaria such as Digitaria ciliaris (DIGAD, southern crabgrass), Digitaria sanguinalis (DIGSA, hairy crabgrass), Digitaria insularis (TRCIN, sourgrass) or Digitaria ischaemum (DIGIS, smooth crabgrass); Echinochloa such as Echinochloa colonum (ECHCO, awnless barnyardgrass), Echinochloa crus-galli (ECHCG, common barnyard grass), Echinochloa crus-pavonis (ECHCV, Gulf cockspurgrass), Echinochloa oryzoides (ECHOR, early barnyardgrass) or Echinochloa phyllogogon (ECHPH, late barnyardgrass); Eleusine such as Eleusine indica (ELEIN, Indian goosegrass); Ischaemum such as Ischaemum rugusom (ISCRU, muraina grass); Leptochloa such as Leptochloa chinensis (LEFCH, Chinese sprangletop), Leptochloa fascicularis (LEFFA, salt-meadow grass), Leptochloa filiformis (LEFPC, thread sprangletop), Leptochloa mucronata (LEFFI, red sprangletop), Leptochloa panicoides (LEFPA, tighthead sprangletop), Leptochloa scabra (LEFSC) or Leptochloa virgata (LEFVI, tropical sprangletop); Lolium such as Lolium multiflorum (LOLMU, Italian ryegrass), Lolium perenne (LOLPE, English ryegrass) or Lolium rigidum (LOLRI, annual rye-grass); Panicum such as Panicum capillare (PANCA, tumble panicgrass), Panicum dichotomiflorum (PANDI, smooth witchgrass), Panicum laevifolium (PANLF, sweet panicgrass) or Panicum miliaceum (PANMI, common millet); Phalaris such as Phalaris minor (PHAMI, lesser canary grass), Phalaris paradoxa (PHAPA, paradoxagrass), Phalaris canariensis (PHACA, canarygrass) or Phalaris brachystachys (PHABR, short-spiked canarygrass); Poa such as Poa annua (POAAN, annual bluegrass), Poa pratensis (POAPR, Kentucky bluegrass) or Poa trivialis (POATR, rough meadowgrass); Rottboellia such as Rottboellia exaltata (ROOEX, guinea-fowl grass); Setaria such as Setaria faberi (SETFA, giant foxtail), Setaria glauca (PESGL, pearl millet), Setaria italic (SETIT, Italian millet), Setaria pumila (SETPU, yellow foxtail), Setaria verticillata (SETVE, bristly foxtail) or Setaria viridis (SETVI, green foxtail); and Sorghum such as Sorghum halepense (SORHA, Johnson grass).

Preferably, the harmful organism is a monocotyledonous weed species selected from the genera Agropyron, Alopecurus, Apera, Avena, Brachiaria, Bromus, Cynodon, Digitaria, Echinochloa, Eleusine, Ischaemum, Leptochloa, Lolium, Panicum, Phalaris, Poa, Rottboellia, and Setaria. More preferably, the harmful organism is selected from the genera Alopecurus, Apera, Avena, Digitaria, Echinochloa, Leptochloa, Lolium, Phalaris, Poa and Setaria. In particular, the harmful organism is selected from the genera Alopecurus, Apera, Avena, Echinochloa, Leptochloa, Lolium, Phalaris and Poa. Most preferably, the harmful organism is selected from the genera Alopecurus, Avena, Lolium and Phalaris.

In another embodiment, the harmful organism is a monocotyledonous weed species selected from the genera Alopecurus, Apera, Avena, Bromus, Echinochloa, Lolium and Setaria.

In another embodiment, the harmful organism is a monocotyledonous weed species selected from the genera Alopecurus, Apera, Lolium and Poa.

In another embodiment, the harmful organism is a monocotyledonous weed species selected from Agropyron repens, Alopecurus myosuroides, Alopecurus aequalis, Apera spica-venti, Avena fatua, Avena sterilis subsp. sterilis, Brachiaria plantaginea, Brachiaria decumbens, Bromus inermis, Bromus sterilis, Bromus tectorum, Bromus arvensis, Bromus secalinus, Bromus hordeacus, Cynodon dactylon, Digitaria ciliaris, Digitaria sanguinalis, Digitaria insularis, Digitaria ischemum, Echinochloa colona, Echinochloa crus-galli, Echinochloa crus-pavonis, Echinochloa erecta, Echinochloa oryzoides, Echinochloa phyllogogon, Eleusine indica, Ischaemum rugusom, Leptochloa chinensis, Leptochloa fascicularis, Leptochloa filliformis, Leptochloa panicoides, Leptochloa scabra, Leptochloa virgata, Lolium multiflorum, Lolium perenne, Lolium rigidum, Panicum capillare, Panicum dichotomiflorum, Panicum laevifolium, Panicum miliaceum, Phalaris minor, Phalaris paradoxa, Phalaris canariensis, Phalaris brachystachys, Poa annua, Poa pratensis, Poa trivialis, Rottboellia exaltata, Setaria faberi, Setaria glauca, Setaria italica, Setaria pumila, Setaria verticillata, and Setaria viridis.

In particular, the monocotyledonous weed species is selected from Alopecurus myosuroides, Alopecurus aequalis, Apera spica-venti, Avena fatua, Avena sterilis subsp. sterilis, Echinochloa crus-galli, Echinochloa oryzoides, Leptochloa chinensis, Lolium multiflorum, Lolium perenne, Lolium rigidum, Phalaris minor, Phalaris paradoxa, Phalaris canariensis, Phalaris brachystachys, Poa annua, Poa pratensis and Poa trivialis, more preferably from Alopecurus myosuroides, Alopecurus aequalis, Apera spica-venti, Avena fatua, Echinochloa grus-galli, Echinochloa oryzoides, Leptochloa chinensis, Lolium multiflorum, Lolium rigidum, Phalaris minor and Poa annua, and most preferably from Alopecurus myosuroides, Avena fatua, Lolium multiflorum, Lolium rigidum and Phalaris minor.

In another embodiment, the monocotyledonous weed species is selected from Alopecurus myosuroides, Apera spica-venti, Avena fatua, Bromus sterilis, Echinochloa crus-galli, Lolium multiflorum and Setaria viridis.

In another embodiment, the monocotyledonous weed species is selected from Alopecurus myosuroides, Apera spica-venti, Lolium multiflorum and Poa annua.

In another embodiment, the harmful organism is a dicotyledonous weeds, in particular broadleaf weeds including, but not limited to, Polygonum species such as Polygonum convolvolus (POLCO, wild buckwheat), Amaranthus species such as Amaranthus albus (AMAAL, tumble pigweed), Amaranthus blitoides (AMABL, mat amaranth), Amaranthus hybridus (AMACH, green pigweed), Amaranthus palmeri (AMAPA, Palmer amaranth), Amaranthus powellii (AMAPO, Powell amaranth), Amaranthus retroflexus (AMARE, redroot pigweed), Amaranthus tuberculatus (AMATU, rough-fruit amaranth), Amaranthus rudis (AMATA, tall amaranth) or Amaranthus viridis (AMAVI, slender amaranth), Chenopodium species such as Chenopodium album (CHEAL, common lambsquarters), Chenopodium ficifolium (CHEFI, fig-leaved goosefoot), Chenopodium polyspermum (CHEPO, many-seeded goosefoot) or Chenopodium hybridum (CHEHY, maple-leaf goosefoot), Sida species such as Sida spinosa L. (SIDSP, prickly sida), Ambrosia species such as Ambrosia artemisiifolia (AMBEL, common ragweed), Acanthospermum species, Anthemis species such as Anthemis arvensis (ANTAR, field chamomile), Atriplex species, Cirsium species, Convolvulus species, Conyza species such as Conyza bonariensis (ERIBO, hairy horseweed) or Conyza canadensis (ERICA, Canada horseweed), Cassia species, Commelina species, Datura species, Euphorbia species, Geranium species such as Geranium dissectum (GERDI, cut-leaf geranium), Geranium pusillium (GERPU, small-flower geranium) or Geranium rotundifolium (GERRT, round-leaved cranesbill), Galinsoga species, Ipomoea species such as Ipomoea hederacea (IPOHE, morningglory), Lamium species, Malva species, Matricaria species such as Matricaria chamomilla (MATCH, wild chamomile), Matricaria discoidea (MATMT, pineapple weed) or Matricaria inodora (MATIN, false chamomille), Sysimbrium species, Solanum species, Xanthium species, Veronica species, Viola species, Stellaria species such as Stellaria media (STEME, common chickweed), Abutilon theophrasti (ABUTH, velvet leaf), Hemp sesbania (Sesbania exaltata Cory, SEBEX, Colorado river hemp), Anoda cristata (ANVCR, cottonweed), Bidens pilosa (BIDPI, common blackjack), Centaurea species such as Centaurea cyanus (CENCY, cornflower), Galeopsis tetrahit (GAETE common hemp nettle), Galium aparine (GALAP, cleavers or goosegrass), Galium spurium (GALSP, false cleavers), Galium tricornutum (GALTC, corn cleavers), Helianthus annuus (HELAN, common sunflower), Desmodium tortuosum (DEDTO, giant beggar weed), Kochia scoparia (KCHSC, mock cypress), Mercurialis annua (MERAN, annual mercury), Myosotis arvensis (MYOAR, field forget-me-not), Papaver rhoeas (PAPRH, common poppy), Salsola kali (SASKA, prickly glasswort), Sonchus arvensis (SONAR, corn sowthistle), Tagetes minuta (TAGMI, Mexican marigold), Richardia brasiliensis (RCHBR, Brazil pusley), cruciferous weeds such as Raphanus raphanistrum (RAPRA, wild radish), Sinapis alba (SINAL, white mustard), Sinapis arvensis (SINAR, wild mustard), Thlaspi arvense (THLAR, fanweed), Descurainia Sophia (DESSO, flixweed), Capsella bursa-pastoris (CAPBP, shepherd's purse), Sisymbrium species such as Sisymbrium officinale (SSYOF, hedge mustard) or Sisymbrium orientale (SSYOR, oriental mustard), Brassica kaber (SINAR wild mustard).

Organism Stress

In another preferred embodiment, the type of the response of the at least one organism is:
(1) target-site resistance of the at least one organism to the treatment with specific treatment parameters, preferably methods or products, or
(2) non-target-site resistance of the at least one organism to the treatment with specific treatment parameters, preferably methods or products, or
(3) response of the at least one organism to the treatment with specific treatment parameters, preferably methods or products, due to at least one of the following stress factors:
a) organism nutrition deficiencies, for example plant nutrition deficiencies,
b) heat stress, for example temperature conditions higher than 30° C.,
c) cold stress, for example temperature conditions lower than 10° C.,
d) drought stress,
e) water stress, for example existence of excessive water, e.g. after heavy rains or floods,
f) exposure to excessive sun light, for example exposure to sun light causing signs of scorch, sun burn or similar signs of irradiation,
g) acidic or alkaline pH conditions in the soil with pH values lower than pH 5 and/or pH values higher than 9,
h) salt stress, for example soil salinity,
i) pollution with chemicals, for example with heavy metals, and/or
j) destructive weather conditions, for example hail, frost, damaging wind.

The response of the at least one organism to the treatment with specific treatment parameters, preferably methods or products, due to at least one of the above stress factors can be preferably phenotypic adaptations such as the development of a thicker cuticula. This can be preferably analysed via specific (genetic) analysis methods such as hyperspectral analysis of the plant (e.g. weed plant), by which also correlations between these specific responses and genetic variants can be found.

For example, it can be derived from the genetic information whether a certain weed species or variety has developed a thicker cuticula as reaction to cold stress, thus a higher dosage of herbicides will be determined as treatment parameter and optionally outputted as control signal to the agricultural equipment.

For example, it can be derived from the genetic information whether a certain weed species has developed target-side resistance to a certain herbicide mode of action class, thus the usage of a different herbicide class or a mixture of different herbicide classes will be determined as treatment parameter and optionally outputted as control signal to the agricultural equipment.

Organism response data are any data relating to the abiotic stress response, biotic stress response, target-site resistance and/or non-target-site resistance of the at least one organism.

Use Case 1: Change in the Resistance of Crop Plants Against Certain Fungi

In another preferred embodiment, the beneficial organism is the agricultural crop plant as such.

In another preferred embodiment, the following computer-implemented method was found: Computer-implemented method for determining at least one of the treatment parameters selected from the group consisting of:
a) at least one time window for a treatment in an agricultural field,
b) at least one method for a treatment in an agricultural field,
c) at least one product for a treatment in an agricultural field,
d) at least one dose rate for a treatment in an agricultural field, and
e) at least one application map for conducting a zone-specific treatment in an agricultural field, comprising the following steps:
(step 1) providing genetic information of at least one organism which existed or is existing or is expected to exist in the agricultural field,
(step 2) at least based on the genetic information of the at least one organism, initiating and/or performing data processing in at least one database and/or database system containing
(i) genetic information data and
(ii) data related to the at least one treatment parameter,
(step 3) outputting the at least one treatment parameter based on the result of the data processing,
wherein the at least one organism is the agricultural crop plant.

In another preferred embodiment, the following computer-implemented method was found: Computer-implemented method for determining at least one of the treatment parameters selected from the group consisting of:
a) at least one time window for a treatment in an agricultural field,
b) at least one method for a treatment in an agricultural field,
c) at least one product for a treatment in an agricultural field,
d) at least one dose rate for a treatment in an agricultural field, and
e) at least one application map for conducting a zone-specific treatment in an agricultural field, comprising the following steps:
(step 1) providing genetic information of at least one organism which existed or is exist-ing or is expected to exist in the agricultural field,
(step 2) at least based on the genetic information of the at least one organism, initiating and/or performing data processing in at least one database and/or database system containing
(i) genetic information data and
(ii) data related to the at least one treatment parameter,
(step 3) outputting the at least one treatment parameter based on the result of the data processing,
wherein the at least one organism is the agricultural crop plant and wherein the genetic information is epigenetic information, information on epigenetic variants or changes, or information on genetic change.

Preferably, the epigenetic information, information on epigenetic variants or changes, or information on gene shifting are information indicative of the status of resistance of agricultural crop plant against harmful organisms (such as fungi).

For example, over several years, agricultural crop plants which were originally resistant against certain harmful organisms (such as fungi) may develop genetic changes (which include epigenetic changes), so that their resistance against these harmful organisms may become lost. Through a genetic analysis, it can be determined whether the resistance of agricultural crop plants against certain harmful organisms are still present. In case the genetic analysis provides the result that such resistance is not present anymore, other treatment parameters can be determined to address this result. For instance, if such result is obtained before the agricultural crop plant has been sown, the treatment parameter to be determined will provide that (i) either other varieties of agricultural crop plants should be sown, or (ii) specific products have to be applied for controlling or targeting the harmful organism to compensate the fact that agricultural crop plant has lost its resistance against such harmful organism. For instance, if such result is obtained after the agricultural crop plant has been sown, the treatment parameter to be determined will provide that specific products have to be applied for controlling or targeting the harmful organism to compensate the fact that agricultural crop plant has lost its resistance against such harmful organism.

In another preferred embodiment (referred to as “resistance-check embodiment”), the following computer-implemented method was found: Computer-implemented method for determining at least one treatment parameter selected from the group consisting of
at least one product for a treatment in an agricultural field, and
at least one dose rate for a treatment in an agricultural field, comprising the following steps:

(step 1) providing genetic information of at least one organism which existed or is existing or is expected to exist in the agricultural field, wherein the at least one organism is an agricultural crop plant and wherein the genetic information includes epigenetic information, information on epigenetic variants or changes, or information on genetic change,

(step 2) at least based on the genetic information of the at least one organism, initiating and/or performing data processing in at least one database and/or database system containing

(i) genetic information data and

(ii) data related to the treatment parameter,

wherein the data processing includes
determining based on the genetic information whether the resistance of the agricultural crop plant against a certain harmful organism is present, such harmful organism being one which existed or is existing or is expected to exist in the agricultural field,
and in case that this resistance is not present and the agricultural crop plant has already been sown, determining a product and/or dose rate usable for controlling or targeting such harmful organism,
and in case that this resistance is not present and the agricultural crop plant has not been sown yet, determining a product and/or dose rate, wherein such product is the seeds of another variety of the agricultural crop plant being resistant against such harmful organism,

(step 3) outputting the at least one treatment parameter based on the result of the data processing, wherein this treatment parameter is preferably outputted as a control signal for an agricultural equipment. Preferably, this resistance check embodiment also includes before (step 1) the following (step 0): taking at least one sample of the at least one organism which existed or is existing or is expected to exist in the agricultural field, conducting a genetic analysis using the at least one sample of the at least one organism, and obtaining therefrom the genetic information of the at least one organism. Preferably, the steps (step 1) and (step 2) and (step 3) of this resistance-check embodiment are carried out in a real-time mode, i.e. preferably less than a minute, more preferably within 10 to 45 seconds, most preferably within 1 to 10 seconds, more preferably within 0.5 to 1 seconds, most preferably within 100 to 500 milliseconds, particularly within 10 to 100 milliseconds. Preferably, the steps (step 0)—if present—and (step 1) and (step 2) and (step 3) of this resistance-check embodiment are carried out in a real-time mode, i.e. preferably less than ten minutes, more preferably less than five minutes, most prefer-ably less than two minutes, particularly more preferably within 10 to 45 seconds, particularly most preferably within 1 to 10 seconds, particularly within 0.5 to 1 seconds, for instance within 100 to 500 milliseconds, for example within 10 to 100 milliseconds.

Use Case 2: Quality Management/Control of Microbials/Bacteria Used as Biological Crop Protection Product

In another preferred embodiment, the beneficial organism is a species of fungi, viruses, viroids, bacteria, protozoa, insects, arachnids, nematodes, mollusks which is applied as biological crop protection product.

In another preferred embodiment, the following computer-implemented method was found: Computer-implemented method for determining at least one of the treatment parameters selected from the group consisting of:
a) at least one time window for a treatment in an agricultural field,
b) at least one method for a treatment in an agricultural field,
c) at least one product for a treatment in an agricultural field,
d) at least one dose rate for a treatment in an agricultural field, and
e) at least one application map for conducting a zone-specific treatment in an agricultural field, comprising the following steps:
(step 1) providing genetic information of at least one organism which existed or is existing or is expected to exist in the agricultural field,
(step 2) at least based on the genetic information of the at least one organism, initiating and/or performing data processing in at least one database and/or database system containing
(i) genetic information data and
(ii) data related to the at least one treatment parameter,
(step 3) outputting the at least one treatment parameter based on the result of the data processing,
wherein the at least one organism is a beneficial organism selected from fungi, viruses, viroids, bacteria, insects, arachnids, nematodes, mollusks, or protozoa, which is applied as biological crop protection product for treatment in an agricultural field.

This method is useful for quality control or quality management—either before or shortly after application—of the beneficial organism selected from fungi, viruses, viroids, bacteria, insects, arachnids, nematodes, or mollusks which is applied as biological crop protection product for treatment in an agricultural field.

For example, a bacteria X to be applied to the agricultural field as biofungicide has developed into another biotype which has a decreased biofungicidal effect or has no biofungicidal effect. Via genetic analysis of the bacteria X before or shortly after application of bacteria X, this fact that this biotype has a decreased biofungicidal effect or has no biofungicidal effect could be found, thus, another treatment parameter using the same biofungicide in a higher dosage or using another biofungicide or another fungicide will be outputted.

In another preferred embodiment (referred to as “biologicals-quality-check embodiment”), the following computer-implemented method was found: Computer-implemented method for determining at least one of the treatment parameters selected from the group consisting of at least one product for a treatment in an agricultural field, and at least one dose rate for a treatment in an agricultural field,
comprising the following steps:
(step 1) providing genetic information of at least one organism, wherein the at least one organism is a beneficial organism selected from fungi, viruses, viroids, bacteria, insects, arachnids, nematodes, mollusks, or protozoa, which is applied or to be applied as biological crop protection product for treatment in an agricultural field,
(step 2) at least based on the genetic information of the at least one organism, initiating and/or performing data processing in at least one database and/or database system containing
(i) genetic information data and
(ii) data related to the at least one treatment parameter,
wherein the data processing includes
   determining based on the genetic information whether the organism has or will have the planned effect or efficacy as biological crop protection product,
   in case the organism does not have or will not have the planned effect or efficacy as biological crop protection product, determining a product and/or dose rate usable for compensating the loss of effect or efficacy of the organism as biological crop protection product,
(step 3) outputting the at least one treatment parameter based on the result of the data processing, wherein this treatment parameter is preferably outputted as a control signal for an agricultural equipment. Preferably, this biologicals-quality-check embodiment also includes before (step 1) the following (step 0): taking at least one sample of the at least one organism, conducting a genetic analysis using the at least one sample of the at least one organism, and obtaining therefrom the genetic information of the at least one organism. Preferably, the steps (step 1) and (step 2) and (step 3) of this biologicals-quality-check embodiment are carried out in a realtime mode, i.e. preferably less than a minute, more preferably within 10 to 45 seconds, most preferably within 1 to 10 seconds, more preferably within 0.5 to 1 seconds, most preferably within 100 to 500 milliseconds, particularly within 10 to 100 milliseconds. Preferably, the steps (step 0)—if present—and (step 1, and (step 2) and (step 3) of this biologicals-quality-check embodiment are carried out in a real-time mode, i.e. preferably less than ten minutes, more preferably less than five minutes, most prefer-ably less than two minutes, particularly more preferably within 10 to 45 seconds, particularly most preferably within 1 to 10 seconds, particularly within 0.5 to 1 seconds, for instance within 100 to 500 milliseconds, for example within 10 to 100 milliseconds.

### DESCRIPTION OF DRAWINGS

FIG. 1 illustrates one example of a distributed computing system 10 for controlling or monitoring a treatment on an agricultural field using the agricultural treatment device 20.

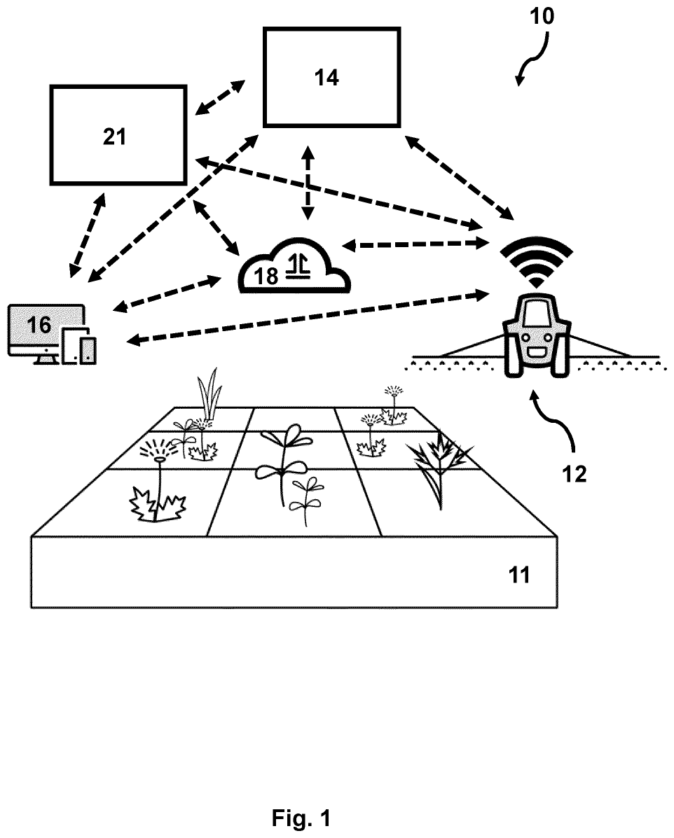

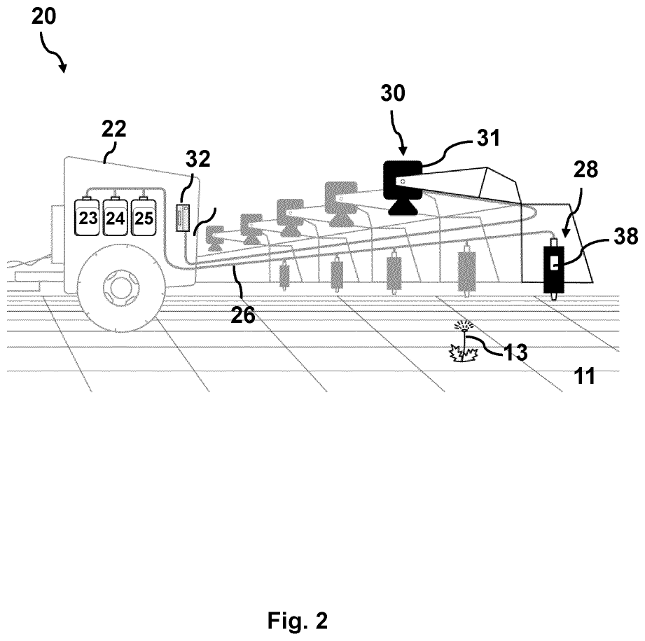

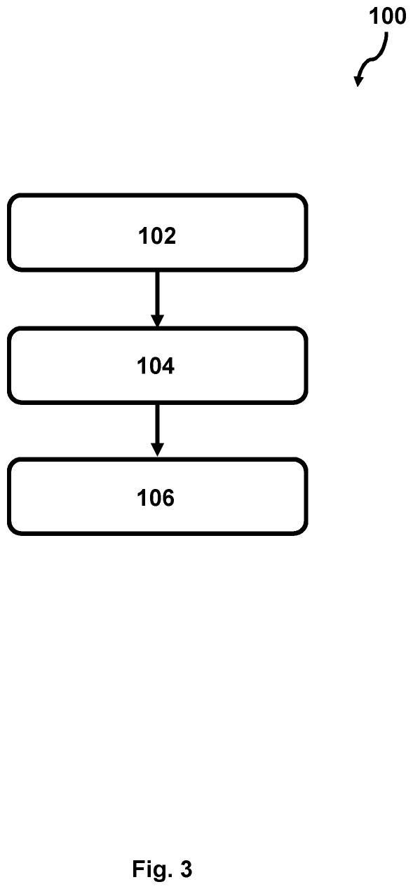

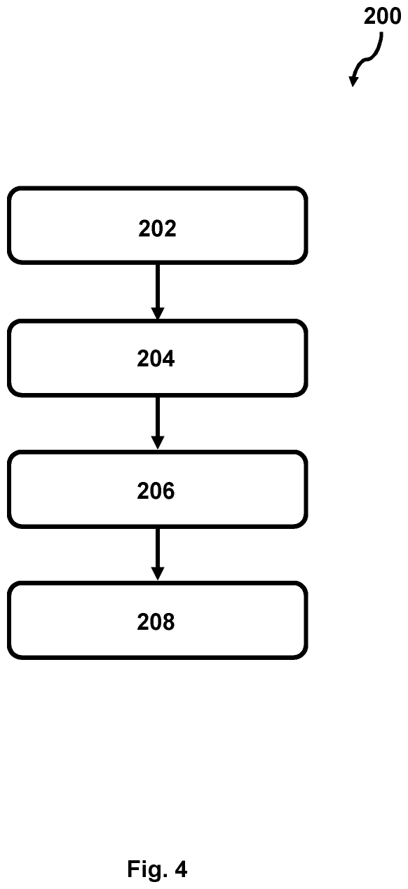

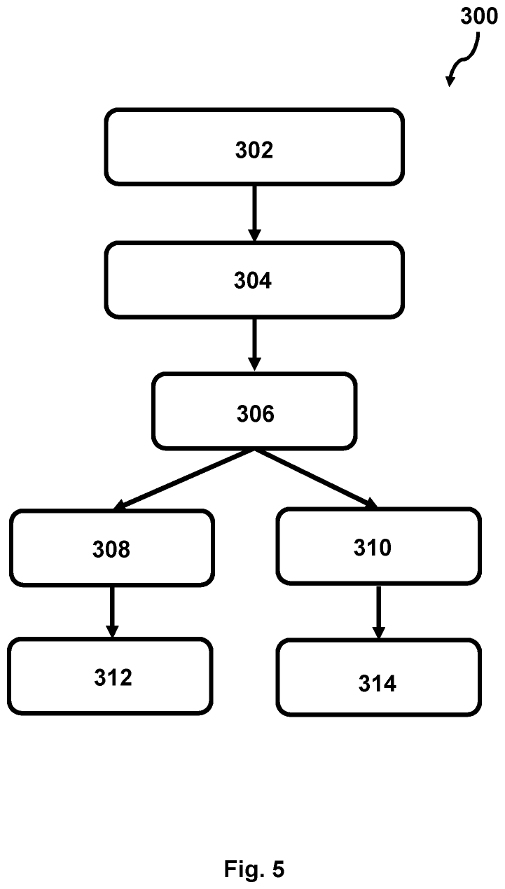

The distributed system 10 is configured for treatment of a field 11 cultivating crops. The field 11 may be any plant or crop cultivation area at a geo-referenced location. As indicated in FIG. 1 by interlines, the field 11 may optionally be divided into two or more sub-areas illustrating zone-specific or location specific specificity. The system 10 may include a distributed computing system with remote computing resources 12, 14, 16, 18, 20. The system 10 may include smart machinery configured to treat the field, such as one or more crop protection treatment device(s) 20 or one or more harvesting device(s), a preparation system 14 configured to control or monitor crop protection treatment, a client device 16 configured to display output data to a user or to collect input data from a user, a data distribution system 18 configured to send or receive data packets and one or more production management system(s) 20 configured to monitor processing of the agricultural product harvested. The field 11 may be treated by use of a crop protection product such as an herbicide, a fungicide, an insecticide or a nematicide.

For a more integrated controlling or monitoring, the system 10 includes a preparation system 14 for generating the treatment control data. The treatment control data may be a data set in a machine-readable format including
at least one field identifier indicating the location of the field 11 and/or field attributes including crop data such as crop type or crop growth stage;
at least one treatment product identifier indicating a treatment product to be applied on the field 11, such as an herbicide, a fungicide, an insecticide or a nematicide;
at least one treatment operation parameter indicating an amount of treatment product to be applied to the field 11; and
at least one treatment time or time window indicating a time for conducting the treatment on the field.

The treatment control data may be provided to the crop protection treatment device 20 prior to or during the treatment. The treatment device 20 may control the application of the treatment product, such as an herbicide, a fungicide, an insecticide or a nematicide, to the field 11 based on the treatment operation parameter and the treatment time or time range. The treatment control data may be spatially resolved in one or more data points by relating the data point to a location or sub-area of the field 11. The treatment control data may include one treatment product identifier associated with the treatment product or product mix to be applied to the field 11. The treatment control data may include more than one treatment product identifier indicating a spatially resolved treatment product map with different treatment products or product mixes to be applied in different locations of the field 11. The treatment control data may include one treatment operation parameter associated with an amount or dosage of treatment product to be applied to the field 11. The treatment control data may include more than one treatment operation parameter indicating a spatially resolved treatment map with different amounts of treatment products to be applied in different locations of the field 11. The treatment control data may include one treatment time or time range associated with the time for conducting the treatment on the field 11. The treatment control data may include more than one treatment time or time window indicating the spatially resolved timing map with different treatment times or time ranges for treating the field 11 in different locations.

The preparation system 14 may include a database or database system which is used in (step 2). Through data processing in this database or database system, treatment parameters such as products may be determined for treating the plants cultivated on the field 11. The preparation system 14 may include an interface configured to receive genetic information from genetic analysis conducted either (real-time) during or prior to treatment on the field 11. The preparation system 14 may for instance include an interface configured to receive data related to treatment parameters such as product data. The preparation system 14 may include an interface configured to send at least one treatment control data (relating to the highest ranked treatment parameter) to the treatment device 20, the client device 16, the data distribution system 18 or the processing system 21. Similar interfaces may be included in the treatment device 20, the client device 16, the data distribution system 18 or the processing system 21 to send or receive respective data packages. In particular, when data is monitored, collected and/or recorded by any treatment device 20, such data may be distributed to one or more of, or to every computing system 14, 16, 18, 20 of the distributed computing system 10.

FIG. 2 illustrates one example of a crop protection treatment device 20 for applying a crop protection product (such as an herbicide, a fungicide, an insecticide or a nematicide) to a field. It is noted that FIG. 2 is merely schematic illustrating main components. The agricultural treatment device 20 may comprise more, less, or different components than shown.

The agricultural treatment device 20 may be part of the machinery 10 (as shown in FIG. 1) and configured to apply the crop protection product on the field 11 or on one or more subareas thereof. The release elements 28 may be configured to apply crop protection product to the field 11. In at least some embodiments, the agricultural treatment device 20 may comprise a boom with multiple release elements 28 arranged along the boom. The release elements 28 may be fixed or may be attached movably along the boom in regular or irregular intervals. Each release element 28 may be arranged together with one or more, preferably separately, controllable valves 38 to regulate treatment product release to the field 11.

One or more tank(s) 23, 24, 25 may be placed in a housing 22 and may be in communication with the release elements 28 through one or more connections 28, which distribute the one or more products (such as an herbicide, a fungicide, an insecticide or a nematicide). Each tank 23, 24, 25 may further comprise a controllable valve to regulate release from the tank 23, 24, 25 to connections 26.

The tank valves and/or the release elements 28 may be communicatively coupled to a control system 32. In the embodiment shown in FIG. 2, the control system 32 is located in a main housing 22 and wired to the respective components. In another embodiment the tank valves or the valves of the release elements 28 may be wirelessly connected to the control system 32. In yet another embodiment more than one control system 32 may be distributed in the housing 22 and communicatively coupled to the tank valves or the valves of the release elements 28.

The control system 32 may be configured to control the tank valves or the valves of the release elements 28 based on the treatment control data. The treatment control data may be a control file or control protocol based on which the agricultural treatment device 20 is controlled during treatment. The control system 32 may comprise multiple electronic modules with instructions, which when executed control the treatment, in particular by controlling the tank release or the release elements 28. One module for instance may be configured to collect data during application on the field 11, e.g. location data. A further module may be configured to receive the control file with the treatment control data. A further module may be configured to derive a control signal from the location data and the control file. Yet further module(s) may be configured to control the tank 23, 24, 25 release and/or release elements 28 based on such derived control signal. Yet further module(s) may be configured to store control and/or monitoring data of the treatment device 20, such as as-applied maps, during treatment execution on the field 11. Yet further module(s) may be configured to provide control and/or monitoring data of the treatment device 20, such as as-applied maps, collected during treatment execution on the field 11 to e.g. the client device 16, the data distribution system 18 or the processing system 21 of FIG. 1.

FIG. 3 and FIG. 4 illustrate the workflow of the embodiments of the present invention.

In FIG. 3, a computer-implemented method (100) for determining at least one of the treatment parameters selected from the group consisting of:
a) at least one time window for a treatment in an agricultural field,
b) at least one method for a treatment in an agricultural field,
c) at least one product for a treatment in an agricultural field,
d) at least one dose rate for a treatment in an agricultural field, and
e) at least one application map for conducting a zone-specific treatment in an agricultural field, comprising the following steps:
(step 1) (102) providing genetic information of at least one organism which existed or is existing or is expected to exist in the agricultural field,
(step 2) (104) at least based on the genetic information of the at least one organism, initiating and/or performing data processing in at least one database and/or database system containing
   (i) genetic information data and
   (ii) data related to the at least one treatment parameter,
(step 3) (106) outputting the at least one treatment parameter based on the result of the data processing,
is shown.

In FIG. 4, a computer-implemented method (200) for determining at least one of the treatment parameters selected from the group consisting of:
a) at least one time window for a treatment in an agricultural field,
b) at least one method for a treatment in an agricultural field,
c) at least one product for a treatment in an agricultural field,
d) at least one dose rate for a treatment in an agricultural field, and
e) at least one application map for conducting a zone-specific treatment in an agricultural field, comprising the following steps:
   (step 1) (202) providing genetic information of at least one organism which existed or is existing or is expected to exist in the agricultural field,
   (step 1a) (204) providing
      (A) agricultural crop data comprising (aa) information about an agricultural crop species grown, sown, planned to be grown, or planned to be sown in the agricultural field, and/or (bb) information about the growth stage of such agricultural crop, or
      (B) weather and/or geographical data relating to the location of the agricultural field, or
      (C) historic treatment data relating to treatments conducted in the agricultural field in the past,
   (step 2) (206) at least based on the genetic information of the at least one organism and based on the agricultural crop data or the weather and/or geographical data or the historic treatment data, initiating and/or performing data processing in at least one database and/or database system containing
      (i) genetic information data and
      (ii) data related to the at least one treatment parameter and
      (iii) data related to agricultural crop data, or data related to weather and/or geographical data, or data related to historic treatment data
   (step 3) (208) outputting the at least treatment parameter based on the result of the data processing, is shown.

In FIG. 5, the computer-implemented method (300) of the “resistance-check embodiment” is shown: In step (302), genetic information of at least one organism which existed or is existing or is expected to exist in the agricultural field is provided, wherein the at least one organism is an agricultural crop plant and wherein the genetic information includes epigenetic information, information on epigenetic variants or changes, or information on genetic change. In step (304), at least based on the genetic information of the at least one organism, data processing in at least one database and/or database system containing
(i) genetic information data and
(ii) data related to the treatment parameter,

is initiated and/or performed.

In step (306) as substep of step (304), the data processing includes determining based on the genetic information whether the resistance of the agricultural crop plant against a certain harmful organism is present, such harmful organism being one which existed or is existing or is expected to exist in the agricultural field. In step (308) as substep of step (304), it has been determined that this resistance is not present and the agricultural crop plant has already been sown, thus a product (for example a fungicide, or an insecticide) and/or dose rate usable for controlling or targeting such harmful organism has been determined. In step (312), this product and/or dose rate has been outputted as treatment parameter based on the result of the data processing, and this treatment parameter has been outputted as control signal for an agricultural equipment. In step (310) as substep of step (304), it has been determined that this resistance is not present and the agricultural crop plant has not been sown yet, thus a product and/or dose rate has been determined, wherein such product is the seeds of another variety of the agricultural crop plant being resistant against such harmful organism. In step (314), this product being the seeds of another variety of the agricultural crop plant and/or the corresponding dose rate has been outputted as treatment parameter based on the result of the data processing, and this treatment parameter has been outputted as control signal for an agricultural equipment.

1. A computer-implemented method for determining at least one of the treatment parameters selected from the group consisting of:
a) at least one time window for a treatment in an agricultural field,
b) at least one method for a treatment in an agricultural field,
c) at least one product for a treatment in an agricultural field,
d) at least one dose rate for a treatment in an agricultural field, and
e) at least one application map for conducting a zone-specific treatment in an agricultural field,
the method comprising:
(step 1) providing genetic information of at least one organism which existed or is existing or is expected to exist in the agricultural field;
(step 2) at least based on the genetic information of the at least one organism, initiating and/or performing data processing in at least one database and/or database system containing
(i) genetic information data, and
(ii) data related to the at least one treatment parameter; and
(step 3) outputting the at least one treatment parameter based on the result of the data processing.

2. The method according to claim 1, further comprising:
(step 1) providing genetic information of at least one organism which existed or is existing or is expected to exist in the agricultural field;
(step 1a) providing:
(A) agricultural crop data comprising (aa) information about an agricultural crop species grown, sown, planned to be grown, or planned to be sown in the agricultural field, and/or (bb) information about the growth stage of such agricultural crop, or
(B) weather and/or geographical data relating to the location of the agricultural field, or
(C) historic treatment data relating to treatments conducted in the agricultural field in the past,
(step 2) at least based on the genetic information of the at least one organism and based on the agricultural crop data or the weather and/or geographical data or the historic treatment data, initiating and/or performing data processing in at least one database and/or database system containing:
(i) genetic information data,
(ii) data related to the at least one treatment parameter, and
(iii) data related to agricultural crop data, or data related to weather and/or geographical data, or data related to historic treatment data; and
(step 3) outputting the at least one treatment parameter based on the result of the data processing.

3. The method according to claim 1, further comprising:
(step 1) providing genetic information of at least one organism which existed or is existing or is expected to exist in the agricultural field,
(step 1a) providing
(A) agricultural crop data comprising (aa) information about an agricultural crop species grown, sown, planned to be grown, or planned to be sown in the agricultural field, and/or (bb) information about the growth stage of such agricultural crop, and
(B) weather and/or geographical data relating to the location of the agricultural field, or
(step 2) at least based on the genetic information of the at least one organism and based on the agricultural crop data and the weather and/or geographical data, initiating and/or performing data processing in at least one database and/or database system containing:
(i) genetic information data,
(ii) data related to the at least one treatment parameter,
(iii) data related to agricultural crop data, and
(iv) data related to weather and/or geographical data; and
(step 3) outputting the at least one treatment parameter based on the result of the data processing.

4. The method according to claim 1, further comprising:
(step 1) providing genetic information of at least one organism which existed or is existing or is expected to exist in the agricultural field,
(step 1a) providing
(A) agricultural crop data comprising (aa) information about an agricultural crop species grown, sown, planned to be grown, or planned to be sown in the agricultural field, and/or (bb) information about the growth stage of such agricultural crop, and
(C) historic treatment data relating to treatments conducted in the agricultural field in the past,
(step 2) at least based on the genetic information of the at least one organism and based on the agricultural crop data and the historic treatment data, initiating and/or performing data processing in at least one database and/or database system containing:
(i) genetic information data,
(ii) data related to the at least one treatment parameter,
(iii) data related to agricultural crop data,
(iv) data related to historic treatment data; and
(step 3) outputting the at least one treatment parameter based on the result of the data processing.

5. The method according to claim 1, further comprising:
(step 1) providing genetic information of at least one organism which existed or is existing or is expected to exist in the agricultural field,
(step 1a) providing:
(B) weather and/or geographical data relating to the location of the agricultural field, and
(C) historic treatment data relating to treatments conducted in the agricultural field in the past,
(step 2) at least based on the genetic information of the at least one organism and based on the weather and/or geographical data and the historic treatment data, initiating and/or performing data processing in at least one database and/or database system containing:
(i) genetic information data,
(ii) data related to the at least one treatment parameter,
(iii) data related to weather and/or geographical data, and
(iv) data related to historic treatment data; and
(step 3) outputting the at least one treatment parameter based on the result of the data processing.

6. The method according to claim 1, further comprising:
(step 1) providing genetic information of at least one organism which existed or is existing or is expected to exist in the agricultural field,
(step 1a) providing:
(A) agricultural crop data comprising (aa) information about an agricultural crop species grown, sown, planned to be grown, or planned to be sown in the agricultural field, and/or (bb) information about the growth stage of such agricultural crop, and
(B) weather and/or geographical data relating to the location of the agricultural field, and
(C) historic treatment data relating to treatments conducted in the agricultural field in the past,
(step 2) at least based on the genetic information of the at least one organism and based on the agricultural crop data and the weather and/or geographical data and the historic treatment data, initiating and/or performing data processing in at least one database and/or database system containing:
(i) genetic information data,
(ii) data related to the at least one treatment parameter,
(iii) data related to agricultural crop data,
(iv) data related to weather and/or geographical data, and
(v) data related to historic treatment data; and
(step 3) outputting the at least one treatment parameter based on the result of the data processing.

7. The method according to claim 1, wherein the treatment parameter is to be outputted as a control signal for an agricultural equipment.

8. The method according to claim 1, further comprising the following step before (step 1):
(step 0) taking at least one sample of the at least one organism which existed or is existing or is expected to exist in the agricultural field, conducting a genetic analysis using the at least one sample of the at least one organism, and obtaining therefrom the genetic information of the at least one organism.

9. The method according to claim 1, further comprising the following step before (step 1):
(step 0) taking at least one sample of the at least one organism which existed or is existing or is expected to exist in the agricultural field, conducting a genetic analysis using the at least one sample of the at least one organism, and obtaining therefrom the genetic information of the at least one organism, wherein the genetic analysis is based on at least one of the technologies selected from the group consisting of nanopore technology, microarray technology, graphene biosensor technology, PCR (polymerase chain reaction) technology, fast PCR technology, and other DNA/RNA amplification technologies such as isothermal amplification.

10. The method according to claim 8, wherein the genetic analysis of the at least one organism is conducted using a portable device operated in the agricultural field.

11. The method according to claim 8, wherein the steps (step 0) and (step 1) and (step 2) and (step 3) are carried out in a real-time mode.

12. The method according to claim 8, wherein the steps (step 0) and (step 1) and (step 2) and (step 3) are carried out in less than two minutes.

13. The method according to claim 8, wherein the treatment parameter is to be outputted as a control signal for an agricultural equipment and wherein the steps (step 0) and (step 1) and (step 2) and (step 3)—including the outputting as control signal for an agricultural equipment—are carried out in less than two minutes.

14. (canceled)

15. The method according to claim 1, wherein the at least one organism is:
a harmful organism selected from the group consisting of: weeds, fungi, viruses, viroids, bacteria, insects, arachnids, nematodes, mollusks, birds, and rodents; or
a beneficial organism selected from the group consisting of: beneficial plants, fungi, viruses, viroids, bacteria, insects, arachnids, nematodes, mollusks, birds, rodents, and protozoa.

16. (canceled)

17. The method according to claim 1, wherein the at least one organism is an agricultural crop species grown, sown, planned to be grown, or planned to be sown in the agricultural field.

18. The method according to claim 1, wherein at least the steps (step 1), (step 2) and (step 3) are carried out in a real-time mode.

19. The method according to claim 1, wherein genetic information data contains organism response data and wherein the data processing includes the determination of the type of response of the at least one organism based on the organism response data.

20. The method according to claim 1, wherein genetic information data contains organism response data and wherein the data processing includes the determination of the type of response of the at least one organism based on the organism response data, and wherein the type of the response of the at least one organism is:
(1) target-site resistance of the at least one organism to the treatment with specific treatment parameters, or
(2) non-target-site resistance of the at least one organism to the treatment with specific treatment parameters, or
(3) response of the at least one organism to the treatment with specific treatment parameters due to at least one of the following stress factors:
a) organism nutrition deficiencies, for example plant nutrition deficiencies,
b) heat stress, for example temperature conditions higher than 30° C.,
c) cold stress, for example temperature conditions lower than 10° C.,
d) drought stress,
e) water stress, for example existence of excessive water, e.g. after heavy rains or floods,
f) exposure to excessive sun light, for example exposure to sun light causing signs of scorch, sun burn or similar signs of irradiation,
g) acidic or alkaline pH conditions in the soil with pH values lower than pH 5 and/or pH values higher than 9,
h) salt stress, for example soil salinity,
i) pollution with chemicals, for example with heavy metals, and/or
j) destructive weather conditions, for example hail, frost, damaging wind.

21. A data processing system comprising one or more processors configured to perform the method according to claim 1.

22. (canceled)

23. A non-transitory computer-readable storage medium comprising instructions which, when executed by a computer, cause the computer to carry out the computer-implemented method according to claim 1.

24. (canceled)

25. (canceled)
